(19) United States
(12) Patent Application Publication
(10) Pub. No.: US 2022/0395541 A1
(43) Pub. Date: Dec. 15, 2022

(54) Methods and Compositions Comprising a Bacteria with Increased Viability and Faster Revivability

(71) Applicant: Pendulum Therapeutics, Inc., San Francisco, CA (US)

(72) Inventors: Nicholas B. Justice, South East Atlanta, GA (US); Magdalena K. Stoeva, Oakland, CA (US); John S. Eid, San Francisco, CA (US)

(21) Appl. No.: 17/787,258

(22) PCT Filed: Dec. 18, 2020
(86) PCT No.: PCT/US2020/066095

§ 371 Date: Jun. 21, 2022

Related U.S. Application Data
(60) Provisional application No. 62/951,772, filed on Dec. 20, 2019.

(51) Int. Cl. A61K 35/74; A61P 3/10; A61P 3/04; A61K 9/00

(52) U.S. Cl. CPC A61K 35/74; A61P 3/10; A61P 3/04; A61K 9/0053

(57) ABSTRACT

Methods to increase viability and revivability of bacteria compared to their wild type counterparts, are disclosed herein. New strains of an anerobic bacteria is also disclosed obtained by the methods described herein. Further, disclosed are compositions comprising these bacteria as well as methods of treatment including the use of the bacteria described.

### CROSS REFERENCE

This application claims priority to U.S. Provisional Patent Application No. 62/951,772, filed Dec. 20, 2019, which is incorporated herein by reference in its entirety.

### FIELD OF THE INVENTION

The present disclosure relates to methods of generating and isolating bacteria with improved revival properties as well as bacteria obtained and isolated by such methods and methods of using such bacteria.

### BACKGROUND OF THE INVENTION

Eubacteria is a genus of gram-positive, non-sporulating, strictly anaerobic bacteria. Culturing and formulating compositions comprising Eubacteria can be difficult due to the bacteria's strict anaerobic demands. The genus Eubacterium includes Eubacterium alactolyticum, Eubacterium saburreum, Eubacterium lentum, Eubacterium limosum, Eubacterium nodatum, Eubacterium brachy, Eubacterium timidum, Eubacterium saphenus, and Eubacterium minutum, and Eubacterium hallii among others.

### BRIEF SUMMARY OF THE INVENTION

Described herein are methods of isolating a bacteria having increased viability when revived from freezing and/or lyophilization. Such methods include culturing the bacteria in anaerobic conditions in/on a vegetable-based growth medium; lyophilizing the bacteria from the culture in the presence of a cryoprotectant; and reviving the lyophilized bacteria. The methods may also include determining that the revived bacteria has increased viability and/or isolating the revived bacteria with the increased viability. Increased viability may be increased viability as compared to a wild-type bacteria being revived from lyophilization and/or freezing.

Further described herein are new strains of anaerobic bacteria, and compositions comprising such bacteria. In some embodiments, bacteria as described herein include the strain as deposited at the ATCC®, under ATCC® deposit number PTA-126295 (herein referred to as EHAL-ALE).

Further included are bacteria comprising one or more of the sequences of SEQ ID NOs: 2, 4, 6, 8, 10, 12, and 14. In still further embodiments, described herein are strains comprising one or more of: a deletion of C at a position corresponding to any one of positions 63 to 71 of SEQ ID No. 1; a deletion of AACAAT at a position corresponding to positions 79 to 84 of SEQ ID No. 3; a replacement of G with an A at a position corresponding to position 713 of SEQ ID No. 5; a deletion of A at a position corresponding to any one of positions 29 to 30 of SEQ ID No. 7; a replacement of G with an A at a position corresponding to any one of positions 209 or 210 of SEQ ID No. 9; a replacement of C with a G at a position corresponding to position 735 of SEQ ID No. 11; or a replacement of G with an A at a position corresponding to any one of positions 1378 or 1379 of SEQ ID No. 13.

In embodiments, any of the bacteria comprising one or more of the sequences of SEQ ID NOs: 2, 4, 6, 8, 10, 12, and 14 or with any one or more mutations of SEQ ID Nos.: 1, 3, 5, 7, 9, 11, and 13, as described above, are collectively termed as EHAL-Mut.

Disclosed herein, in some aspects, is a method of isolating a bacteria having increased viability when revived from lyophilization, the method comprising: culturing the bacteria in anaerobic conditions in/on a vegetable-based growth medium; lyophilizing the bacteria from the culture in the presence of a cryoprotectant; reviving the lyophilized bacteria; determining that the revived bacteria has increased viability; isolating the revived bacteria with the increased viability; and wherein the increased viability is as compared to a wild-type bacteria being revived from lyophilization.

Disclosed herein, in some aspects, is a bacteria as deposited at ATCC accession number 126295.

Disclosed herein, in some aspects, is a bacteria comprising at least one of SEQ ID No., 2, 4, 6, 8, 10, 12 or 14.

Disclosed herein, in some aspects, is a bacteria comprising at least one of: (a) a deletion of C at a position corresponding to any one of positions 63 to 71 of SEQ ID No. 1; (b) a deletion of AACAAT at a position corresponding to positions 79 to 84 of SEQ ID No. 3; (c) a replacement of G with an A at a position corresponding to position 713 of SEQ ID No. 5; (d) a deletion of A at a position corresponding to any one of positions 29 to 30 of SEQ ID No. 7; (e) a replacement of G with an A at a position corresponding to any one of positions 209 or 210 of SEQ ID No. 9; (f) a replacement of C with a G at a position corresponding to position 735 of SEQ ID No. 11; or (g) a replacement of G with an A at a position corresponding to any one of positions 1378 or 1379 of SEQ ID No. 13.

In some embodiments, culturing the bacteria comprises culturing the bacteria to obtain least 1×10\hat{ }5 AFUs/g of the culture. In some embodiments, culturing the bacteria comprises measuring the growth of the bacteria at regular time intervals. In some embodiments, the regular time intervals are every 10 minutes, every 20 minutes, every 30 minutes, every 1 hour, every 4 hours, or every 6 hours. The method according to claim 1, wherein the vegetable-based medium is essentially free of any animal or dairy components or derivatives thereof. In some embodiments, the vegetable-based medium comprises peptone, yeast extract, and glucose. In some embodiments, the bacterium is as deposited at ATCC® accession number 126295. In some embodiments, the bacterium comprises at least one of SEQ ID No., 2, 4, 6, 8, 10, 12 or 14. In some embodiments, the bacterium comprises at least one of: (a) a deletion of C at a position corresponding to any one of positions 63 to 71 of SEQ ID No. 1; (b) a deletion of AACAAT at a position corresponding to positions 79 to 84 of SEQ ID No. 3; (c) a replacement of G with an A at a position corresponding to position 713 of SEQ ID No. 5; (d) a deletion of A at a position corresponding to any one of positions 29 to 30 of SEQ ID No. 7; (e) a replacement of G with an A at a position corresponding to any one of positions 209 or 210 of SEQ ID No. 9; (f) a replacement of C with a G at a position corresponding to position 735 of SEQ ID No. 11; or (g) a replacement of G with an A at a position corresponding to any one of positions 1378 or 1379 of SEQ ID No. 13. In some embodiments, the bacteria has at least 95% 16s rDNA sequence homology to the full length 16s rRNA of any one of Eubacterium hallii DSM 3353, Akkermansia muciniphila ATCC BAA¬83, Clostridium butyricum ATCC 1939, Clostridium beijerinckii ATCC 5174 or combinations thereof. In some embodiments, the bacteria is Eubacterium hallii. In some embodiments, the bacteria belongs to the genus Clostridia, Eubacteria, Bifidobacteria, Anaerostipes, Coprococcus, Bacteroides, Blautia, Ruminococcus, Faecalibacterium, Oscillospira, Streptococcus, or Roseburiai. In some embodiments, the bacteria is selected from the group consisting of Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and all combinations thereof.

Disclosed herein, in some aspects, is a composition comprising any one or more of bacteria as in the preceding embodiments.

In some embodiments, the composition comprises at least 10\hat{ }5 AFUs/g of each of the one or more additional bacteria. In some embodiments, the composition further comprises a prebiotic. In some embodiments, the prebiotic is selected from the group consisting of inulin, green banana, reishi, tapioca, oats, pectin, potato or extracts thereof. In some embodiments, at least one of the one or more additional bacteria is an anaerobic bacteria. In some embodiments, the bacteria is lyophilized. In some embodiments, the composition comprises at least 10\hat{ }5 AFUs/g of the bacteria. In some embodiments, the bacteria is not viable or is dead. In some embodiments, the composition is in the form of a pill, a capsule, a lozenge, a food bar, or a gummy ball. In some embodiments, the bacteria is pasteurized. In some embodiments, the method comprises administering to the subject the bacteria of any one of claims 1-12. In some embodiments, the method further comprises administering to the subject at least one another anaerobic bacteria. In some embodiments, the subject suffers from a gut disorder. In some embodiments, the gut disorder is irritable bowel syndrome, inflammatory bowel disease, a stomach ulcer, pouchitis, Helicobacter pylori infection, or diarrhea. In some embodiments, the subject suffers from, or has a risk of, an insulin resistance-based disorder or an insulin sensitivity-based disorder. In some embodiments, the subject suffers from, or has a risk of, Type-2-diabetes. In some embodiments, the subject is a mammal. In some embodiments, the mammal is a human. In some embodiments, the administration is an oral administration.

Disclosed herein, in some aspects, is a composition comprising a bacteria having a revival rate that is at least 25% faster than E. hallii deposited at DSM 3353, wherein the bacteria has at least 97% sequence identity to the full length of a 16S rRNA sequence of E. hallii as deposited at DSM 3353, and wherein the revival rate is measured by an assay comprising the steps of: (a) providing a lyophilized bacteria and a lyophilized E. hallii deposited at DSM 3353; (b) suspending the lyophilized bacteria and the lyophilized E. hallii in a vegetable medium; (c) incubating the suspension for at least 24 hours in 37° C.; (d) measuring bacterial growth in regular time intervals of at least 5 minutes at an OD600; and (e) determining revival rate of the bacteria and the E. hallii deposited as DSM 3353 based on the measured bacterial growth in step (d) herein.

In some embodiments, the bacteria is deposited at ATCC® with an accession number 126295. In some embodiments, the bacteria comprises at least one of SEQ ID No., 2, 4, 6, 8, 10, 12 or 14. In some embodiments, the bacteria comprises at least one of: (a) a deletion of C at a position corresponding to any one of positions 63 to 71 of SEQ ID No. 1; (b) a deletion of AACAAT at a position corresponding to positions 79 to 84 of SEQ ID No. 3; (c) a replacement of G with an A at a position corresponding to position 713 of SEQ ID No. 5; (d) a deletion of A at a position corresponding to any one of positions 29 to 30 of SEQ ID No. 7; (e) a replacement of G with an A at a position corresponding to any one of positions 209 or 210 of SEQ ID No. 9; (f) a replacement of C with a G at a position corresponding to position 735 of SEQ ID No. 11; or (g) a replacement of G with an A at a position corresponding to any one of positions 1378 or 1379 of SEQ ID No. 13. In some embodiments, the media is free of any animal product or dairy product or derivatives thereof. In some embodiments, the media is a PYG media further comprising salt, buffer, and vitamins. In some embodiments, the method further comprises one or more additional bacterial strains. In some embodiments, the one or more additional bacteria strains are selected from the group consisting of Akkermansia muciniphila, Clostridium butyricum, Clostridium beijerinckii, and all combinations thereof. In some embodiments, the one or more additional bacteria strains are selected from the group consisting of Akkermansia muciniphila ATCC BAA-83, Clostridium butyricum ATCC 1939, Clostridium beijerinckii ATCC 5174 or combinations thereof. In some embodiments, the one or more additional bacteria strains are selected from the group consisting of Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and all combinations thereof. In some embodiments, each of the one or more additional bacteria strains has a 16S rRNA sequence comprising at least about 95% sequence identity to the full length of a 16S rRNA sequence of a microbe selected from the group consisting of: Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof. In some embodiments, the one or more additional bacteria strains belong to the genus of Clostridia, Eubacteria, Bifidobacteria, Anaerostipes, Coprococcus, Bacteroides, Blautia, Ruminococcus, Faecalibacterium, Oscillospira, Streptococcus, or Roseburiail. In some embodiments, the composition comprises at least 10\hat{ }5 AFUs/g of each of the one or more additional bacteria. In some embodiments, the composition further comprises a prebiotic. In some embodiments, the prebiotic is selected from the group consisting of inulin, green banana, reishi, tapioca, oats, pectin, potato or extracts thereof. In some embodiments, at least one of the one or more additional bacteria is an anaerobic bacteria. In some embodiments, the composition comprises at least 10\hat{ }5 AFUs/g of the bacteria. In some embodiments, the composition is in the form of a pill, a capsule, a lozenge, a food bar, or a gummy ball. In some embodiments, the composition further comprises an enteric coating. In some embodiments, the enteric coating is Eudragit FS30D.

### BRIEF SUMMARY OF FIGURES

FIG. 1: Is a graphical representation of certain methods of selecting microbes as described herein.

FIG. 2: Depicts the results of a viability comparison between two Eubacterium hallii strains. The figure shows E. hallii 3353 (OG) strain generally has lower viability compared with the EHAL-ALE strain.

FIG. 3a: Depicts growth curves for five independent replicate comparisons (reps 1-5) of revivability between E. hallii DSM 3353 and EHAL-ALE.

FIG. 3b: Depicts Rep. 2 of the independent replicate comparison demonstrating the lag phase difference between E.hallii DSM 3353 AND EHAL-ALE

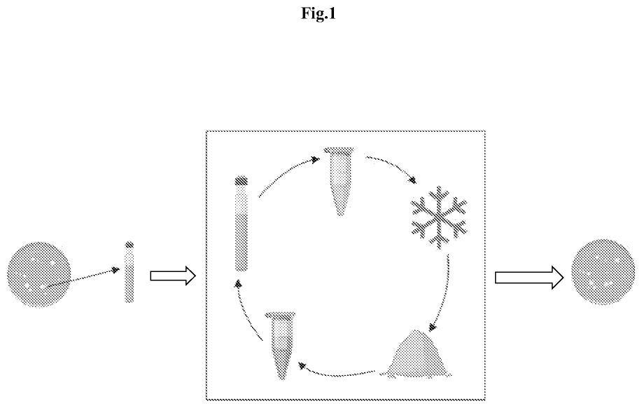

### DETAILED DESCRIPTION OF THE INVENTION

### Strain Deposit

The following biological material has been deposited under the terms of the Budapest Treaty with the American Type Culture Collection (ATCC®), Patent Depository—P0071744 at 10801 University Boulevard Manassas, Va. 20110, and given the following accession number:

| Name/Designation | Patent Deposit Number | Patent Deposit No. |
|---|---|---|
| Eubacterium hallii | WB-STR-0029 | PTA- 126295 |

### A. BACTERIA

Described herein are new strains of anaerobic bacteria. In some embodiments, bacteria as described herein include the strain as deposited at the ATCC®, under ATCC® deposit number PTA-126295 (herein referred to as EHAL-ALE).

Further included are bacteria comprising one or more of the sequences of SEQ ID NOs: 2, 4, 6, 8, 10, 12, and 14. In still further embodiments, described herein are strains comprising one or more of: a deletion of C at a position corresponding to any one of positions 63 to 71 of SEQ ID No. 1; a deletion of AACAAT at a position corresponding to positions 79 to 84 of SEQ ID No. 3; a replacement of G with an A at a position corresponding to position 713 of SEQ ID No. 5; a deletion of A at a position corresponding to any one of positions 29 to 30 of SEQ ID No. 7; a replacement of G with an A at a position corresponding to any one of positions 209 or 210 of SEQ ID No. 9; a replacement of C with a G at a position corresponding to position 735 of SEQ ID No. 11; or a replacement of G with an A at a position corresponding to any one of positions 1378 or 1379 of SEQ ID No. 13.

Any of the bacteria comprising one or more of the sequences of SEQ ID NOs: 2, 4, 6, 8, 10, 12, and 14 or with any one or more mutations of SEQ ID Nos.: 1, 3, 5, 7, 9, 11, and 13, as described above, are collectively referred to herein as EHAL-Mut.

EHAL-ALE and any one or more of EHAL-Mut are collectively termed as bacteria of the present disclosure.

Further described herein are compositions comprising any one or more of EHAL-ALE or an EHAL-Mut. In additional embodiments, described herein are compositions comprising EHAL-ALE and any one or more of EHAL-Mut.

In certain embodiments, the composition comprises EHAL-ALE or an EHAL-Mut as the sole bacteria therein.

In particular embodiments, compositions comprising EHAL-ALE and/or one or more EHAL-mut further comprise a prebiotic. The prebiotic can be at least one or a combination of a plurality of prebiotics selected from inulin, green banana, reishi, tapioca, oats, pectin, potato and extracts thereof.

### B. ADDITIONAL BACTERIA

Embodiments of compositions described herein include but are not limited to, compositions comprising EHAL-ALE and/or an EHAL-Mut in combination with one more other bacteria. Examples of bacteria which may be combined in a composition with EHAL-ALE and/or one or more EHAL-mut include, but are not limited to, Akkermansia muciniphila, Clostridium butyricum, Clostridium beijerinckii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

In certain embodiments, compositions comprising EHAL-ALE and/or one or more EHAL-mut further comprise bacteria of a genus selected from the group consisting of Clostridia, Eubacteria, Bifidobacteria, Anaerostipes, Coprococcus, Bacteroides, Blautia, Ruminococcus, Faecalibacterium, Oscillospira, Streptococcus, and Roseburia.

In one embodiment, a composition comprises of EHAL-ALE and/or one or more of EHAL-Mut further comprises one or more additional bacteria strains are selected from the group consisting of Akkermansia muciniphila ATCC BAA83, Clostridium butyricum ATCC 1939, Clostridium beijerinckii ATCC 5174, and all combinations thereof.

In one embodiment, a composition comprises of EHAL-ALE and/or one or more of EHAL-Mut further comprises one or more additional bacteria strains are selected from the group consisting of Akkermansia muciniphila, Clostridium butyricum, Clostridium beijerinckii and all combinations thereof.

In one embodiment, the additional bacteria strains in compositions therein are anaerobic bacteria. In another embodiment, the additional bacteria are not viable. In still other embodiments, the additional bacteria are lyophilized. In other embodiments, the additional bacteria are lyophilized and viable.

In another embodiment, the present disclosure provides compositions comprising EHAL-ALE and/or one or more EHAL-Mut, and at least one bacteria with a 16S rRNA sequence comprising at least about an 85%, 87%, 90%, 92%, 94%, 95%, 96%, 97%, 98%, 99%, or 100% sequence identity to the full length of a 16S rRNA sequence of a microbe selected from the group consisting of: Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and all combinations thereof.

### C. PROPERTIES OF BACTERIA

In embodiments, phenotypic properties or bacteria described herein may comprise one or more of viability, revivability, ability to make proteins, vitamins, intermediates, fatty acids, short chain fatty acids (SCFA), or other metabolites of interest.

Properties of viability and revivability of frozen or lyophilized strains are measured and compared to a base strain. Such a base strain may be one that is deposited with a certified depositing institution or any other starting material.

In a particular embodiment, properties of revivability and viability of lyophilized strain(s) of E.hallii are measured and compared to the properties of E.hallii DSM 3353.

a. Viability

Viability, as defined herein, is the measure of live cells. Specifically, viability is defined as the ability to multiply via binary fission under controlled conditions measured in Colony Forming Units (CFUs) that measure viability by culturing the microbes and counting only viable cells. Another unit is Active Fluorescence Units (AFUs) that measure viability based on flow cytometer results that correlate cell wall integrity to live cells.

Viability may be measured using flow cytometry (BD Accuri™ C6). Such flow cytometry measurements include staining the cell mixture and detecting the fluorescence of each cell as it flows by. The staining of the cell mixture can be done using any of the stains that preferentially stain cells, especially live and dead cells, or just dead cells. Examples of such stains include, but are not limited to, Thiazole Orange, Propidium Iodide, 7-AAD, DAPI, SYTO 9, fixable dyes like Zombie Aqua™, Zombie Green™, Zombie NIR™, Zombie Red™, Zombie Violet™, Zombie UV™, and Zombie Yellow™, or any combinations thereof. In one embodiment, the stain used herein for processes of this disclosure is a combination of thiazole orange and Propidium Iodide.

In further embodiments, the bacteria of the present disclosure are in a viable, lyophilized state. In some embodiments, the bacteria of the present disclosure are not viable.

In embodiments, one or more of the bacteria in any of the compositions described herein may be in a lyophilized state. Further, one or more of the bacteria in any of the compositions described herein may be viable or non-viable.

In some embodiments, the bacteria of the present disclosure are present at a concentration of approximately 1×10³ to 1×10¹⁴ Active Fluorescence Units (AFU)/g, inclusive of any numbers within the range, whereas in other embodiments the concentrations are approximately 1×10⁹ to 1×10¹³ AFU/g, approximately 1×10⁵ to 1×10⁷ AFU/g, or approximately 1×10⁸ to 1×10⁹ AFU/g, inclusive of any numbers within the range.

In certain embodiments, compositions of the present disclosure comprise at least 10\hat{ }5 CFUs of each strain per gram. In a further embodiment, compositions comprise at least 10\hat{ }6 CFUs/g, or at least 10\hat{ }7 CFUs/g, or at least 10\hat{ }8 CFUs/g, or at least 10\hat{ }9 CFUs/g, or at least 10\hat{ }10 CFUs/g, or at least 10\hat{ }11 CFUs/g, or at least 10\hat{ }12 CFUs/g of at least one strain in the composition. In other embodiments, wherein there are more than one strain, each strain may have a viability (measured in CFUs or AFUs) different from the other strains in the composition.

In some embodiments, the bacteria of the present disclosure may revive at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% faster than the wild type counterparts of the strain under similar conditions.

In some embodiments, EHAL-ALE is at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% more viable as compared to E. hallii DSM 3353.

In other embodiments, any one or more of EHAL-Mut is at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% more viable as compared to E. hallii DSM 3353.

In a preferred embodiment, EHAL-ALE is at least 25% more viable compared to E. hallii DSM 3353. In another preferred embodiment, any one or more of EHAL-Mut is at least 25% more viable compared to E. hallii DSM 3353.

b. Revivability

In certain embodiments, bacteria having increased viability when revived from lyophilization or freezing as compared to wild-type bacteria of the same species are disclosed. This property of having the ability to actively multiply under suitable environmental conditions, once suspended in a favorable vegetable media is termed as revivability. The time it takes for bacteria to achieve exponential growth from a non-growth phase is also known as a lag phase, and the time correlated with this phase provides a measure of revivability of a bacteria. Revivability is defined herein as the time it takes for a frozen or lyophilized bacteria to revive, i.e., to get to reach the exponential growth phase when suspended in a suitable growth media under suitable conditions. Shorter the revival time, the better the revivability.

The bacteria of the present disclosure have a revivability much faster than the wild type counterparts or the additional microbes, as alluded to above. The bacteria may revive at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% faster than the wild type counterparts of the strain under similar conditions.

In certain embodiments, methods of isolating bacteria having increased viability when revived from lyophilization as compared to wild-type bacteria of the same species being revived from lyophilization are described. Such methods include culturing the bacteria in anaerobic conditions in/on a vegetable-based growth medium, lyophilizing the bacteria from the culture in the presence of a cryoprotectant, and reviving the lyophilized bacteria. The method may be repeated until bacteria having desired revivability is obtained. Such methods may further include determining that the revived bacteria has increased viability, and isolating the revived bacteria with the increased viability.

In a particular embodiment, properties of revivability of the single-colony lyophilized strain(s) of E.hallii are measured and compared to the properties of E.hallii DSM 3353.

In some embodiments, any one or more of EHAL-Mut revives at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% faster as compared to E. hallii DSM 3353. In a preferred embodiment, any one or more of EHAL-Mut revives at least 80% faster than E. hallii DSM 3353.

In some embodiments, EHAL-ALE revives at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% faster as compared to E. hallii DSM 3353. In a preferred embodiment, EHAL-ALE revives at least 80% faster than E. hallii DSM 3353.

c. SCFA Production

In embodiments, bacteria of the present disclosure have improved short-chain fatty acid (SCFA) production capabilities. SCFAs are defined as fatty acids with less than 6 carbon atoms, derived from intestinal microbial fermentation of indigestible foods. Examples of SCFA include, but are not limited to acetate, butyrate, propionate, formate, isobutyrate, valerate, isovalerate or combination thereof. In particular embodiments, the SCFA is butyrate.

The term “improved” may be construed as a better performance in terms of quantity or quality of the metabolite produced. Improved can also be construed as a quantity higher than or lower than the previously obtained quantity from the wild type counterparts of the bacteria presented in this disclosure. In some instances, the bacteria of the present disclosure can produce quantitatively more SCFA than the wild type counterparts. In other instances, the bacteria of the present disclosure can produce quantitatively lower amounts of SCFA than the wild type counterparts.

In some embodiments EHAL-ALE produces at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% higher SCFA than the wild type counterpart E.hallii DSM 3353. In a preferred embodiment, the EHAL-ALE produces at least 25% more butyrate than E.hallii DSM-3353.

In some embodiments, any one or more of the EHAL-Mut bacteria produces at least 5%, at least 10%, at least 20%, at least 25%, at least 30%, at least 35%, at least 40%, at least 45%, at least 50%, at least 55%, at least 60%, at least 65%, at least 70%, at least 75%, at least 80%, at least 85%, at least 90%, or at least 95% higher SCFA than E.hallii DSM 3353. In a preferred embodiment, the EHAL-Mut produces at least 25% more butyrate than E.hallii DSM-3353.

### D. METHODS OF USE

In further embodiments, the present disclosure provides methods of treating a disorder. Such a disorder may be a gut-based disorder or any other disorder based on an imbalance of bacterial population in the gut. By way of non-limiting example, such a population may be an E.hallii population in the gut. Examples of such disorders include, but are not limited to, irritable bowel syndrome, inflammatory bowel disease, a stomach ulcer, pouchitis, Helicobacter pylori infection, diarrhea, type-1-diabetes mellitus, type-2-diabetes mellitus, and/or obesity.

In embodiments, compositions described herein may include a pharmaceutically acceptable carrier suitable for oral administration to a mammal, for example, as a powdered food supplement, a variety of pelletized formulations, or a liquid formulation. In some embodiments, the composition is in the form of a pill, a capsule, a lozenge, a food bar, and/or a gummy ball. In some embodiments, the composition is in the form of a suppository, or an injection for delivery to a mammal.

### E. METHODS OF MAKING

Embodiments include methods of generating bacteria having an increase revival rate from lyophilization and or freezing. Such bacteria may be obtained by a selection process that takes a parent culture through iterative cycles including the steps of growing in media, pelleting with cryoprotectant, freezing, lyophilizing, and resuspending in media. Bacteria on which the methods described herein may be performed include, but are not limited to, Eubacterium hallii DSM 3353, Akkermansia muciniphila ATCC BAA83, Clostridium butyricum ATCC 1939, Clostridium beijerinckii ATCC 5174, Akkermansia muciniphila, Eubacterium hallii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium hallii, Eubacterium ventriosum, Roseburia faeccis, Roseburia hominis, Roseburia intestinalis. In another embodiment, a bacteria that has a 16S rRNA sequence comprising at least about 95% sequence identity to the full length of a 16S rRNA sequence of any of the microbes alluded to here, may be subjected to such methods for generating a bacteria with an increased revival rate.

In a further embodiment, bacteria belonging to any genus of Clostridia, Eubacteria, Bifidobacteria, Anaerostipes, Coprococcus, Bacteroides, Blautia, Ruminococcus, Faecalibacterium, Oscillospira, Streptococcus, or Roseburiai may be subjected to such methods for generating a bacteria having an increased revival rate.

In some embodiments, bacteria are grown in any suitable medium; some non-limiting examples include PYG, RCM, GYT veg, BHI, nutrient media, minimal media, selective media, and differential media. In a particular embodiment, the media is a vegetable-based media that is free of any animal or dairy based ingredients or derivatives. In another embodiment, the media is a meat-free media that is free of any animal-derived. In an embodiment, the media used is a liquid PYG (Peptone-Yeast-Glucose) media with components as shown in Table 1 here:

TABLE 1
Composition of PYG vegetable media

| | | |
|---|---|---|
| Sodium Chloride | 0.08 | g |
| Sodium Bicarbonate | 0.4 | g |
| Potassium phosphate monobasic | 0.04 | g |
| Magnesium Sulfate Heptahydrate | 0.02 | g |
| Potassium Phosphate, Dibasic | 2.04 | g |
| Calcium Chloride | 0.02 | g |
| Dextrose | 5 | g |
| Cysteine-HCl | 0.5 | g |
| Yeast Extract | 10 | g |
| HiVeg Extract | 5 | g |
| HiVeg Peptone #1 | 5 | g |
| HiVeg Peptone #3 | 5 | g |
| Tween 80 | 1 | ml |
| AntiFoam | 50 | ul |
| Water | 1 | L |

Any of the media, as described herein, may further comprise trace elements of at least one of salts, vitamins, minerals, or buffers. Some of the added vitamins are listed in Table 2 below. In embodiments the pH of the media may range from 6.5-7.5. In further embodiments, the pH of the media may be outside of 6.5-7.5.

TABLE 2
Composition of vitamins in the media

| Vitamins | mg/L |
|---|---|
| D-biotin | 0.2 |
| Ca-pantothenate | 2.5 |
| myoinositol | 20 |
| p-aminobenzoic acid | 0.5 |
| pyridoxine hydrochloride | 5 |
| riboflavine | 0.5 |
| thiamine dichloride | 10 |
| vitamin B12 | 0.2 |
| nicotinic acid | 5 |

Cryoprotectants useful in the present method include, but are not limited to, lactate, trehalose, glycerol, DMSO, propylene glycol, 2-methyl-2, 4-pentanediol, methanamide, glycerophospholipids, proline, sorbitol, Diethyl glycol, sucrose, glucose, and polymers like polyvinyl alcohol, PEG, hydroxyethyl starch, skim milk, tapioca, Polyvinyl-propiline, Inulin, Methylcellulose, Sodium Alginate, Gum Arabic, Propylene Glycol, or Xylitol and all possible combinations thereof. In embodiments, a cryoprotectant is added to the bacteria before freezing.

In particular embodiments, the bacterial pellet is resuspended in a cryoprotectant. Following resuspension, the cryo-suspended bacteria pellet is frozen. For example, the pellet may be flash-frozen in liquid nitrogen. Other freezing techniques that a person of ordinary skill in the art may employ here—including cryogenic (e.g., in ethanol and dry ice, or carbon dioxide), mechanical freezing, or flash-freezing, are contemplated as part of this disclosure.

In certain embodiments, the mixture of cells and cryoprotectant is lyophilized. Such lyophilization may occur over a period of 12-48 h. Lyophilization may be performed under a vacuum and at a temperature below freezing. A non-limiting example of lyophilization conditions is 0.008 mbar and a temperature of −84° C. Other lyophilization conditions that may be altered include the surface area of the product exposed to sublimation, controlling the pressure and temperature of the lyophilization chamber, and altering the amount of heat applied to the product both during sublimation throughout the primary drying phase and throughout desorption in the secondary drying phase.

In a particular embodiment, the bacteria pellet is lyophilized in a LabConco FreeZone 2.5Plus lyophilizer for 8-24 hr at −84° C. and a vacuum pressure of 0.008 to 0.1 mbar.

Lyophilized or frozen bacteria are then revived. In embodiments, the bacteria is revived by placing the bacteria in a culture media and incubating under growth conditions. In a particular embodiment, bacteria in a proportion of ˜0.01 mg/mL are revived by placing the lyophilized cells in liquid PYG medium (as defined above) and allowing the culture to grow for 12-20 h at a temperature suitable for bacterial growth. This process selects for the cells that are capable of reviving rapidly from freezing or lyophilization. The process may then be repeated through additional cycles to further select a bacterial population.

At the end of each cycle, the bacteria are revived, frozen or lyophilized and grown in media under suitable conditions. In particular embodiments a single colony of the bacteria or a sample of bacteria is revived and lyophilized and grown in a liquid PYG media. The number of cycles a selected colony may go through may be at least 5 cycles, at least 10 cycles, at least 15 cycles, at least 20 cycles, at least 25 cycles, at least 30 cycles, at least 35 cycles, at least 40 cycles, at least 45 cycles, at least 50 cycles, at least 55 cycles, at least 60 cycles, at least 65 cycles, at least 70 cycles, at least 75 cycles, at least 80 cycles, at least 85 cycles, at least 90 cycles, at least 95 cycles, at least 100 cycles, at least 105 cycles, at least 110 cycles, at least 115 cycles, at least 120 cycles, at least 125 cycles, at least 130 cycles, at least 135 cycles, at least 140 cycles, at least 145 cycles, or at least 150 cycles. Each cycle may present one or more challenges to the bacteria. Examples of such challenges include, but are not limited to, changes in growth conditions, nutrient medium ingredients, temperature, pressure, oxygen conditions to the process of isolation, freezing and/or lyophilization, and/or revival of microbes as described herein.

In particular embodiment, a microbe of Eubacterium hallii that goes through 22 cycles of microbial selection is then grown on PYG-agar, and a single colony is isolated, named EHAL-ALE, and deposited in ATCC® as ATCC® 126295.

### F. COMPOSITIONS

Provided herein are compositions that comprise one or more bacteria of the present disclosure. Such compositions may be administered to a subject. In certain embodiments, such compositions may be administered as therapeutics or as a dietary supplement. One or more bacteria of the present disclosure, as described herein, may be used to create a pharmaceutical formulation comprising an effective amount of a bacteria for treating a subject. The microorganisms may be in any formulation known in the art. Some non-limiting examples of such formulations include topical, capsule, pill, enema, liquid, injection formulations, and the like. In some embodiments, the one or more strains disclosed herein may be included in a food or beverage product, cosmetic, or nutritional supplement.

In some embodiments, the subject is a mammal. Mammal, as used herein, refers to any mammal, including but not limited to, human, mouse, cat, rat, dog, sheep, monkey, goat, rabbit, hamster, horse, cow or pig. In a preferred embodiment, the mammal is human.

The composition may include one or more active ingredients. Active ingredients include, but are not limited to, those selected from the group consisting of: antibiotics, prebiotics, probiotics, glymays (e.g., as decoys that would limit specific bacterial/viral binding to the intestinal wall), bacteriophages, microorganisms and the like.

In some embodiments, the composition comprises a prebiotic. The prebiotic may be as a combination or as a single ingredient. In some embodiments, the prebiotic may be a source of starch, mucin, fructo-oligosaccharides, pectin, gums, glucans, xylans, arabinogalactan, seaweed polysaccharides, or derivatives thereof. Some examples of such sources are inulin, green banana, reishi, tapioca, oats, pectin, raw or cooked potato, maize, rice, rice bran, cereals, porcine or human sources of mucin, derivatives of L-threonine, chicory root, agave, artichoke, dandelion, lemon peel, apple peel, berries, guar gum, xanthan gum, acacia chia, barley, sorghum, corn, larch, arabinex, kelp, dulse or any combinations or derivatives thereof. In some embodiments, the prebiotic is inulin. Inulin serves as an energy source for the microbial composition.

A composition may be administered by a suitable method for delivery to any part of the gastrointestinal tract of a subject including oral cavity, mouth, esophagus, stomach, duodenum, small intestine regions including duodenum, jejunum, ileum, and large intestine regions including cecum, colon, rectum, and anal canal. In some embodiments, the composition is formulated for delivery to the ileum and/or colon regions of the gastrointestinal tract.

In some embodiments, administration of a composition occurs orally, for example, through a capsule, pill, powder, tablet, gel, or liquid, designed to release the composition in the gastrointestinal tract. In some embodiments, administration of a composition occurs by injection, for example, for a composition comprising butyrate, propionate, acetate, and short chain fatty acids. In some embodiments, the administration of a composition occurs by application to the skin, for example, cream, liquid, or patch. In some embodiments, administration of a composition occurs by a suppository and/or by enema. In some embodiments, a combination of administration routes is utilized.

Microbial compositions may be formulated as a dietary supplement. Microbial compositions may be incorporated with vitamin supplements. Microbial compositions may be formulated in a chewable form such as a probiotic gummy. Microbial compositions may be incorporated into a form of food and/or drink. Non-limiting examples of food and drinks where the microbial compositions may be incorporated include, for example, bars, shakes, juices, infant formula, beverages, frozen food products, fermented food products, and cultured dairy products such as yogurt, yogurt drink, cheese, acidophilus drinks, and kefir.

A composition of the disclosure may be administered as part of a fecal transplant process. A composition may be administered to a subject by a tube, for example, nasogastric tube, nasojejunal tube, nasoduodenal tube, oral gastric tube, oral jejunal tube, or oral duodenal tube. A composition may be administered to a subject by colonoscopy, endoscopy, sigmoidoscopy, and/or enema.

In some embodiments, the microbial composition is formulated such that the one or more microbes may replicate once they are delivered to the target habitat (e.g. the gut). In one non-limiting example, the microbial composition is formulated in a pill. Such a pill may have a shelf life of at least 1, 2, 3, 4, 5, 6, 7, 8, 9, 10, 11, or 12 months. In another non-limiting example, the microbial composition is formulated so that the microbes may reproduce once they are in the gut. In some embodiments, the EHAL-ALE or any one or more of EHAL-Mut may be formulated with other additives in a pill that may be administered orally to an individual, such that the pill does not disintegrate until the pill reaches the intestine of the individual. In some embodiments, other components may be added to aid the shelf life of the microbial composition. In some embodiments, EHAL-ALE or any one or more of EHAL-Mut may be formulated in a manner that it is able to survive in a non-natural environment. For example, a microbe that is native to the gut may not survive in an oxygen rich environment. To overcome this limitation, the microbe may be formulated to reduce or eliminate the exposure to oxygen. Other strategies to enhance the shelf-life of microbes may include other microbes (e.g. i whereby one or more strains in the composition improves the survival of one or more of the other strains in the composition).

In some embodiments, EHAL-ALE and/or any one or more of EHAL-Mut is lyophilized and formulated as a composition, as provided herein. Such a composition may be formulated as a powder, tablet, enteric-coated capsule (e.g. for delivery to ileum/colon), or pill that may be administered to a subject by any suitable route. In some embodiments, the lyophilized formulation may be mixed with a saline or other solution prior to administration.

In some embodiments, wherein the composition comprises of a EHAL-ALE and/or any one or more of EHAL-Mut, wherein the bacteria is completely viable (comprising 100% live population of cells), is partially viable (comprising <50% of live population of cells) or is mostly non-viable (comprising >50% of dead population of cells).

In some embodiments, a microbial composition is formulated for oral administration, for example, as an enteric-coated capsule or pill, for delivery of the contents of the formulation to the ileum and/or colon regions of a subject.

I. Capsule Ingredients

In some embodiments, the microbial composition is formulated for oral administration. In some embodiments, the microbial composition is formulated as an enteric coated pill or capsule for oral administration. In some embodiments, the microbial composition is formulated for delivery of the microbes to the ileum region of a subject. In some embodiments, the microbial composition is formulated for delivery of the microbes to a colon region (e.g. upper and/or lower colon) of a subject. In some embodiments, the microbial composition is formulated for delivery of the microbes to the ileum and colon regions of a subject.

In an embodiment, a composition comprising EHAL-ALE is administered as a capsule for oral delivery.

In another embodiment, a composition comprising any one or more of EHAL-Mut is administered as a capsule for oral delivery. In a further embodiment, any of EHAL-ALE or EHAL-Mut is co-administered with any other anaerobic bacteria in a single capsule or pill.

In an embodiment, the size of the capsule is size 0, although the capsule size may be any size that is suitable for administration to a subject. Further, in the embodiment, the capsule has an enteric coating. An enteric coating may protect the contents of the oral formulation from the acidity of the stomach and provide delivery to the ileum and/or upper colon regions. Non-limiting examples of enteric coatings include pH sensitive polymers (e.g., eudragit FS30D), methyl acrylate-methacrylic acid copolymers, cellulose acetate succinate, hydroxypropyl methyl-cellulose phthalate, hydroxypropyl methyl-cellulose acetate succinate (e.g., hypromellose acetate succinate), polyvinyl acetate phthalate (PVAP), methyl methacrylate-methacrylic acid copolymers, shellac, cellulose acetate trimellitate, sodium alginate, zein, other polymers, fatty acids, waxes, shellac, plastics, and plant fibers. In some embodiments, the enteric coating is formed by a pH sensitive polymer. In some embodiments, the enteric coating is formed by eudragit FS30D.

The enteric coating may be designed to dissolve at any suitable pH. In some embodiments, the enteric coating is designed to dissolve at a pH greater than about pH 6.5 to about pH 7.0. In some embodiments, the enteric coating is designed to dissolve at a pH greater than about pH 6.5. In some embodiments, the enteric coating is designed to dissolve at a pH greater than about pH 7.0. The enteric coating may be designed to dissolve at a pH greater than: 5, 5.1, 5.2, 5.3, 5.4, 5.5, 5.6, 5.7, 5.8, 5.9, 6, 6.1, 6.2, 6.3, 6.4, 6.5, 6.6, 6.7, 6.8, 6.9, 7, 7.1, 7.2, 7.3, 7.4, or 7.5 pH units.

In some embodiments, the administration of a formulation of the disclosure may be preceded by, for example, colon cleansing methods such as colon irrigation/hydrotherapy, enema, administration of laxatives, dietary supplements, dietary fiber, enzymes, and magnesium or derivatives thereof.

Formulations provided herein may include the addition of one or more agents to the therapeutics or cosmetics in order to enhance stability and/or survival of the microbial formulation. Non-limiting example of stabilizing agents include genetic elements, glycerin, ascorbic acid, skim milk, lactose, tween, alginate, xanthan gum, carrageenan gum, mannitol, palm oil, and poly-L-lysine (POPL).

II. Microbial Formulations and Compositions in Microbial Consortia

Compositions as disclosed here may comprise of a single microbial species or a combination of microbes. In one embodiment, the formulation comprises of only EHAL-ALE. In one embodiment, the formulation comprises of any one or more of EHAL-Mut. In another embodiment, the formulation comprises of EHAL-ALE and any one or more of EHAL-Mut.

In another embodiment, formulations comprising at least two microbial population, one of which is the bacteria EHAL-ALE is disclosed. The second microbe may be any one or more of EHAL-Mut or any of the species selected from the group consisting of: Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

In another embodiment, the composition as disclosed herein, comprises of one or more recombinant microbes or microbes that have been genetically modified. In other embodiments, one or more microbes are not modified or recombinant. In some embodiments, the composition comprises microbes that may be regulated, for example, a microbe comprising an operon or promoter to control microbial growth. Microbes, as disclosed herein, may be produced, grown, or modified using any suitable methods, including recombinant methods.

In an embodiment, the population of the bacteria that may be administered as a part of the compositions as disclosed here, may comprise of any one or more of the species selected from the group consisting of: EHAL-ALE, any one or more of EHAL-Mut, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and any combination thereof.

In some embodiments, provided are therapeutic compositions to treat a metabolic disorder comprising a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: EHAL-ALE, any one or more of EHAL-Mut, Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combination thereof.

In some embodiments, the metabolic disorder may be selected from insulin-resistance based disorders, insulin-sensitivity based disorders, type-2-diabetes, obesity, Crohn's disorders, or Inflammatory bowel disorders.

In some embodiments, provided are therapeutic compositions to treat type-2-diabetes or obesity comprising a purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: EHAL-ALE, one or more of EHAL-Mut, Eubacterium hallii DSM 3353, Akkermansia muciniphila ATCC BAA83, Clostridium butyricum ATCC 1939, Clostridium beijerinckii ATCC 5174, Akkermansia muciniphila, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Lactobacillus acidophilus, Lactobacillus brevis, Lactobacillus bulgaricus, Lactobacillus casei, Lactobacillus caucasicus, Lactobacillus fermentum, Lactobacillus helveticus, Lactobacillus lactis, Lactobacillus plantarum, Lactobacillus reuteri, Lactobacillus rhamnosus, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combination thereof.

In some embodiments, provided are therapeutic compositions to treat type-2-diabetes or obesity comprising a purified microorganism population comprising a bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of EHAL-ALE.

In some embodiments, provided are therapeutic compositions to treat type-2-diabetes or obesity comprising a purified microorganism population comprising a bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of any one or more of EHAL-Mut.

In some embodiments, provided are therapeutic compositions to treat type-2-diabetes or obesity comprising a purified microorganism population comprising EHAL-ALE.

In some embodiments, provided are therapeutic compositions to treat type-2-diabetes or obesity comprising a purified microorganism population comprising any one or more of EHAL-Mut.

In some embodiments, provided are therapeutic compositions to treat a metabolic disorder comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: EHAL-ALE, any one or more of EHAL-Mut, Akkermansia muciniphila, Bifidobacterium adolescentis, Bifidobacterium infantis, Bifidobacterium longum, Clostridium beijerinckii, Clostridium butyricum, Clostridium indolis, Faecalibacterium prausnitzii, and any combination thereof.

In some embodiments, provided are therapeutic compositions to treat a metabolic disorder comprising an isolated and/or purified microorganism population consisting of bacteria with at least about: 70%, 75%, 80%, 85%, 87%, 90%, 91%, 92%, 93%, 94%, 95%, 96%, 97%, 98%, 99%, 99.5%, or 100% sequence identity to the 16SrRNA and/or 23S rRNA of a microorganism selected from the group consisting of: EHAL-ALE, any one or more of EHAL-Mut, Eubacterium hallii DSM 3353, Akkermansia muciniphila ATCC BAA83, Clostridium butyricum ATCC 1939, Clostridium beijerinckii ATCC 5174, Akkermansia muciniphila, Bifidobacterium adolescentis, Bifidobacterium infantis, Bifidobacterium longum, Clostridium beijerinckii, Clostridium butyricum, Clostridium indolis, Faecalibacterium prausnitzii, and any combination thereof.

In some embodiments, provided are therapeutic compositions to treat a metabolic disorder comprising an isolated and/or purified microorganism population comprising bacteria selected from the group consisting of: EHAL-ALE, any one or more of EHAL-Mut, Akkermansia muciniphila, Clostridium beijerinckii and Clostridium butyricum.

In one embodiment, provided are therapeutic compositions to treat a metabolic disorder comprising: EHAL-ALE, any one or more of EHAL-Mut, and further comprising one or more additional bacteria strains selected from the group consisting of: Akkermansia muciniphila ATCC BAA83, Clostridium butyricum ATCC 1939, Clostridium beijerinckii ATCC 5174, and all combinations thereof.

In some embodiments, provided are therapeutic compositions to treat a metabolic disorder comprising EHAL-ALE and Akkermansia muciniphila.

In some embodiments, provided are therapeutic compositions to treat Type-2-diabetes or Type-1-diabetes comprising EHAL-ALE and Akkermansia muciniphila.

In some embodiments, provided are therapeutic compositions to treat a metabolic disorder comprising any one or more of EHAL-Mut and Akkermansia muciniphila.

In some embodiments, provided are therapeutic compositions to treat Type-2-diabetes or Type-1-diabetes comprising any one or more of EHAL-Mut and Akkermansia muciniphila.

In some embodiments, provided are therapeutic compositions to treat a metabolic disorder comprising an isolated and/or purified microorganism population comprising bacteria selected from the group consisting of: EHAL-ALE, Akkermansia muciniphila, Bifidobacterium adolescentis, Bifidobacterium infantis, Bifidobacterium longum, Clostridium beijerinckii, Clostridium butyricum, Clostridium indolis, and any combination thereof.

In some embodiments, a therapeutic consortium comprises Akkermansia muciniphila, Bifidobacterium adolescentis, Bifidobacterium infantis, Clostridium beijerinckii, Clostridium butyricum, and EHAL-ALE.

In some embodiments, a therapeutic consortium comprises Akkermansia muciniphila and EHAL-ALE.

In some embodiments, a therapeutic consortium comprises Akkermansia muciniphila and any one or more of EHAL-Mut.

A therapeutic composition may comprise at least 1, at least 2, at least 3, at least 4, at least 5, at least 6, at least 7, at least 8, at least 9, at least 10, at least 11, at least 12, at least 13, at least 14, at least 15, at least 16, at least 17, at least 18, at least 19, at least 20, at least 21, at least 22, at least 23, at least 24, at least 25, at least 26, at least 27, at least 28, at least 29, at least 30, at least 31, at least 32, at least 33, at least 34, at least 35, at least 36, at least 37, at least 38, at least 39, at least 40, at least 45, or at least 50, or at least 75, or at least 100 types of bacteria. A therapeutic composition may comprise at most 1, at most 2, at most 3, at most 4, at most 5, at most 6, at most 7, at most 8, at most 9, at most 10, at most 11, at most 12, at most 13, at most 14, at most 15, at most 16, at most 17, at most 18, at most 19, at most 20, at most 21, at most 22, at most 23, at most 24, at most 25, at most 26, at most 27, at most 28, at most 29, at most 30, at most 31, at most 32, at most 33, at most 34, at most 35, at most 36, at most 37, at most 38, at most 39, at most 40, at most 45, or at most 50, or at most 75, or at most 100 types of bacteria.

In some embodiments, combining one or more microbes in a therapeutic composition or consortia may provide a synergistic effect when administered to the individual. For example, administration of a first microbe may be beneficial to a subject and administration of a second microbe may be beneficial to a subject but when the two microbes are administered together to a subject, the benefit is greater than either benefit alone.

Different types of microbes in a therapeutic composition may be present in the same amount or in different amounts. For example, the ratio of two bacteria in a therapeutic composition may be about 1:1, 1:2, 1:5, 1:10, 1:25, 1:50, 1:100, 1:1000, 1:10,000, or 1:100,000.

III. Compositions Comprising Probiotics

In an embodiment, a composition comprising one single microbe as a probiotic is disclosed. In another embodiment, a composition comprising consortia of bacteria, comprising at least two different bacteria is disclosed. Apart from the probiotic, the composition, may comprise a prebiotic, an antibiotic, or a combination of active agents as described herein.

In some embodiments, a composition is administered before, during, and/or after treatment with an antimicrobial agent such as an antibiotic. For example, the composition may be administered at least about 1 hour, 2 hours, 5 hours, 12 hours, 1 day, 3 days, 1 week, 2 weeks, 1 month, 6 months, or 1 year before and/or after treatment with an antibiotic. The formulation may be administered at most 1 hour, 2 hours, 5 hours, 12 hours, 1 day, 3 days, 1 week, 2 weeks, 1 month, 6 months, or 1 year before and/or after treatment with an antibiotic.

In some embodiments, the composition as disclosed herein, is administered during, after, and/or before with an anti-diabetic agent like insulin or a drug like metformin. For example, the formulation may be administered at least about 1 hour, 2 hours, 5 hours, 12 hours, 1 day, 3 days, 1 week, 2 weeks, 1 month, 6 months, or 1 year before and/or after treatment with the anti-diabetic agent.

In some embodiments, a composition is administered before, during, and/or after food intake by a subject. In some embodiments, the composition is administered with food intake by the subject. In some embodiments, the composition is administered with (e.g., simultaneously) with food intake.

In some embodiments, the composition is administered before food intake by a subject. In some embodiments, the composition is more effective or potent at treating a metabolic disorder when administered before food intake. For example, the composition may be administered about 1 minute, about 2 minutes, about 3 minutes, about 5 minutes, about 10 minutes, about 15 minutes, about 30 minutes, about 45 minutes, about 1 hour, about 2 hours, about 3 hours, about 4 hours, about 5 hours, about 6 hours, about 7 hours, about 8 hours, about 9 hours, about 10 hours, about 12 hours, or about 1 day before food intake by a subject.

In some embodiments, the composition is administered after food intake by the subject. In some embodiments, the composition is more effective or potent at treating a metabolic disorder when administered after food intake. For example, the composition may be administered at least about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject. For example, the formulation may be administered at most about 1 minute, 2 minutes, 3 minutes, 5 minutes, 10 minutes, 15 minutes, 30 minutes, 45 minutes, 1 hour, 2 hours, 3 hours, 5 hours, 10 hours, 12 hours, or 1 day after food intake by a subject.

Compositions provided herein include those suitable for oral including buccal and sub-lingual, intranasal, topical, transdermal, transdermal patch, pulmonary, vaginal, rectal, suppository, mucosal, systemic, or parenteral including intramuscular, intraarterial, intrathecal, intradermal, intraperitoneal, subcutaneous, and intravenous administration or in a form suitable for administration by aerosolization, inhalation or insufflation.

In some embodiments, the composition may be a therapeutic composition, a pharmaceutical composition, or a dietary supplement composition.

A therapeutic composition, as disclosed herein, may include carriers and excipients (including but not limited to buffers, carbohydrates, lipids, mannitol, proteins, polypeptides or amino acids such as glycine, antioxidants, bacteriostats, chelating agents, suspending agents, thickening agents and/or preservatives), metals (e.g., iron, calcium), salts, vitamins, minerals, water, oils including those of petroleum, animal, vegetable or synthetic origin, such as peanut oil, soybean oil, mineral oil, sesame oil and the like, saline solutions, aqueous dextrose and glycerol solutions, flavoring agents, coloring agents, detackifiers and other acceptable additives, adjuvants, or binders, other pharmaceutically acceptable auxiliary substances as required to approximate physiological conditions, such as pH buffering agents, tonicity adjusting agents, emulsifying agents, wetting agents and the like. Examples of excipients include starch, glucose, lactose, sucrose, gelatin, malt, rice, flour, chalk, silica gel, sodium stearate, glycerol monostearate, talc, sodium chloride, dried skim milk, glycerol, propylene, glycol, water, ethanol and the like.

Non-limiting examples of pharmaceutically-acceptable excipients suitable for use in the disclosure include granulating agents, binding agents, lubricating agents, disintegrating agents, sweetening agents, glidants, anti-adherents, anti-static agents, surfactants, antioxidants, gums, coating agents, coloring agents, flavouring agents, dispersion enhancer, disintegrant, coating agents, plasticizers, preservatives, suspending agents, emulsifying agents, plant cellulosic material and spheronization agents, and any combination thereof.

Non-limiting examples of pharmaceutically acceptable excipients can be found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), each of which is incorporated by reference in its entirety.

A therapeutic composition may be substantially free of preservatives. In some applications, the composition may contain at least one preservative.

A therapeutic composition may be encapsulated within a suitable vehicle, for example, a liposome, a microsphere, or a microparticle. Microspheres formed of polymers or proteins may be tailored for passage through the gastrointestinal tract directly into the blood stream. Alternatively, the compound may be incorporated and the microspheres, or composite of microspheres, and may be implanted for slow release over a period of time ranging from days to months.

A therapeutic composition may be formulated as a sterile solution or suspension. The therapeutic compositions may be sterilized by conventional techniques or may be sterile filtered. The resulting aqueous solutions may be packaged for use as is, or lyophilized. The lyophilized preparation of the microbial composition may be packaged in a suitable form for oral administration, for example, capsule or pill. In one embodiment, the capsule may comprise of enteric polymers that do not disintegrate until after the capsule exits the stomach of the subject.

In one embodiment, the capsule ingredient may comprise of methyl cellulose, hydroxypropyl methylcellulose phthalate, propylene glycol and derivatives of such polymers.

The compositions may be administered topically and may be formulated into a variety of topically administrable compositions, such as solutions, suspensions, lotions, gels, pastes, medicated sticks, balms, creams, and ointments. Such pharmaceutical compositions may contain solubilizers, stabilizers, tonicity enhancing agents, buffers and preservatives.

The compositions may also be formulated in rectal compositions such as enemas, rectal gels, rectal foams, rectal aerosols, suppositories, jelly suppositories, or retention enemas, containing conventional suppository bases such as cocoa butter or other glycerides, as well as synthetic polymers such as polyvinylpyrrolidone, PEG, and the like. In suppository forms of the compositions, a low-melting wax such as a mixture of fatty acid glycerides, optionally in combination with cocoa butter, may be used.

In practicing the methods of treatment or use provided herein, therapeutically effective amounts of the microbial compositions described herein are administered in pharmaceutical compositions to a subject having a disease or condition to be treated. In some embodiments, the subject is a mammal such as a human. A therapeutically effective amount may vary widely depending on the severity of the disease, the age and relative health of the subject, potency of the formulation, and other factors. Subjects may be, for example, humans, elderly adults, adults, adolescents, pre-adolescents, children, toddlers, infants, or neonates. A subject may be a patient. A subject may be an individual enrolled in a clinical study. A subject may be a laboratory animal, for example, a mammal.

Pharmaceutical compositions may be formulated using one or more physiologically acceptable carriers comprising excipients and auxiliaries, which facilitate processing of the microorganisms into preparations that may be used pharmaceutically. Formulation may be modified depending upon the route of administration chosen. Pharmaceutical compositions described herein may be manufactured in a conventional manner, for example, by means of conventional mixing, dissolving, granulating, vitrification, spray-drying, lyophilizing, drageemaking, levigating, encapsulating, entrapping, emulsifying or compression processes.

In some embodiments, the pharmaceutical composition is manufactured in a dry form, for example, by spray-drying or lyophilization. In some embodiments, the formulation is prepared as a liquid capsule to maintain the liquid form of the microbes.

Compositions provided herein may be stored at any suitable temperature. The formulation may be stored in cold storage, for example, at a temperature of about −80° c., about −20° c., about −4° c., or about 4° c. The storage temperature may be, for example, about 0° c., about 1° c., about 2° c., about 3° c., about 4° c., about 5° c., about 6° c., about 7° c., about 8° c., about 9° c., about 10° c., about 12° c., about 14° c., about 16° c., about 20° c., about 22° c., or about 25° c. In some embodiments, the storage temperature is between about 2° c. to about 8° c. storage of microbial compositions at low temperatures, for example from about 2° c. to about 8° c., may keep the microbes alive and increase the efficiency of the composition, for example, when present in a liquid or gel formulation. Storage at freezing temperature, below 0° c., with a cryoprotectant may further extend stability.

The pH of the composition may range from about 3 to about 12. The pH of the composition may be, for example, from about 3 to about 4, from about 4 to about 5, from about 5 to about 6, from about 6 to about 7, from about 7 to about 8, from about 8 to about 9, from about 9 to about 10, from about 10 to about 11, or from about 11 to about 12 pH units.

The pH of the composition may be, for example, about 3, about 4, about 5, about 6, about 7, about 8, about 9, about 10, about 11, or about 12 pH units. The pH of the composition may be, for example, at least 3, at least 4, at least 5, at least 6, at least 7, at least 8, at least 9, at least 10, at least 11 or at least 12 pH units. The pH of the composition may be, for example, at most 3, at most 4, at most 5, at most 6, at most 7, at most 8, at most 9, at most 10, at most 11, or at most 12 pH units. If the pH is outside the range desired by the formulator, the pH may be adjusted by using sufficient pharmaceutically acceptable acids and bases. In some embodiments, the pH of the composition is between about 4 and about 6.

Pharmaceutical compositions containing microbes described herein may be administered for prophylactic and/or therapeutic treatments. In therapeutic applications, the compositions may be administered to a subject already suffering from a disease or condition, in an amount sufficient to cure or at least partially arrest the symptoms of the disease or condition, or to cure, heal, improve, or ameliorate the condition. Microbial compositions may also be administered to lessen a likelihood of developing, contracting, or worsening a condition. Amounts effective for this use may vary based on the severity and course of the disease or condition, previous therapy, the subject's health status, weight, and response to the drugs, and the judgment of the treating physician.

Multiple therapeutic agents may be administered in any order or simultaneously. If simultaneously, the multiple therapeutic agents may be provided in a single, unified form, or in multiple forms, for example, as multiple separate pills. The composition may be packed together or separately, in a single package or in a plurality of packages. One or all of the therapeutic agents may be given in multiple doses. If not simultaneous, the timing between the multiple doses may vary as much as about a month.

Compositions described herein may be administered before, during, or after the occurrence of a disease or condition, and the timing of administering the composition may vary. For example, the microbial composition may be used as a prophylactic and may be administered continuously to subjects with a propensity to conditions or diseases in order to lessen the likelihood of the occurrence of the disease or condition. The microbial compositions may be administered to a subject during or as soon as possible after the onset of the symptoms. The administration of the microbial compositions may be initiated within the first 48 hours of the onset of the symptoms, within the first 24 hours of the onset of the symptoms, within the first 6 hours of the onset of the symptoms, or within 3 hours of the onset of the symptoms. The initial administration may be via any route practical, such as by any route described herein using any formulation described herein. A microbial composition may be administered as soon as is practicable after the onset of a disease or condition is detected or suspected, and for a length of time necessary for the treatment of the disease, such as, for example, from about 1 month to about 3 months. The length of treatment may vary for each subject.

In an aspect, compositions may be administered in combination with another therapy, for example, immunotherapy, chemotherapy, radiotherapy, anti-inflammatory agents, anti-viral agents, anti-microbial agents, and anti-fungal agents.

In another aspect, compositions may be packaged as a kit. In some embodiments, a kit includes written instructions on the administration/use of the composition. The written material may be, for example, a label. The written material may suggest conditions methods of administration. The instructions provide the subject and the supervising physician with the best guidance for achieving the optimal clinical outcome from the administration of the therapy. The written material may be a label. In some embodiments, the label may be approved by a regulatory agency, for example the U.S. Food and Drug Administration (FDA), the European Medicines Agency (EMA), or other regulatory agencies.

IV. Methods of Treatment

The disclosure provides methods of treatment of a subject. The subject may be suffering from or may be predisposed to symptoms related to disorders that originate from a disbalance of gut microbiome. Altering the composition of a microbiome in such a subject may have desired health consequences. Compositions of the present disclosure may be administered as a therapeutic and/or a dietary supplement for treating a health condition. Treatments designed to alter the host microbiome(s) may result in a reduction of patient symptoms, prevention of disease, and or treatment of the disease or health condition. For example, modification of the gut microbiome may reduce the risk for health conditions such as metabolic disorders.

Therefore, the present disclosure provides methods for the restoration of a microbial habitat of a subject to a healthy state. The method may comprise microbiome correction and/or adjustment including for example, replenishing native microbes, removing pathogenic microbes, administering prebiotics, and growth factors necessary for microbiome survival. In some embodiments, the method also comprises administering antimicrobial agents such as antibiotics.

Based on the microbiome profile, the present disclosure provides methods for generalized-treatment recommendation for a subject as well as methods for subject-specific treatment recommendation. Methods for treatments may comprise one of the following steps: determining a first ratio of a level of a subject-specific microbiome profile to a level of a second microbiome profile in a biological sample obtained from at least one subject, detecting a presence or absence of a disease in the subject based upon the determining, and recommending to the subject at least one generalized or subject-specific treatment to ameliorate disease symptoms.

The diseases that may be treated include those in which the alteration of the gut microbiome is beneficial to a subject suffering from the disease. Specifically, the gut microbiome of a subject may be scarce on the population of at least one live, active gut bacteria selected from the group consisting of: Akkermansia muciniphila, Clostridium butyricum, Clostridium beijerinckii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Eubacterium hallii, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

In one embodiment, the gut microbe that are in lower levels, in a subject suffering from or predisposed to disorders as mentioned above, is Eubacterium hallii. In another embodiment, the gut microbe that are in lower levels, in a subject suffering from or predisposed to disorders as mentioned above, is Akkermansia muciniphila. In another embodiment, the gut microbe that are in lower levels, in a subject suffering from or predisposed to disorders as mentioned above belongs to the genus Clostridia, Eubacteria, Bifidobacteria, Anaerostipes, Coprococcus, Bacteroides, Blautia, Ruminococcus, Faecalibacterium, Oscillospira, Streptococcus, and Roseburia.

In another embodiment, the subject has more than one gut microbe in levels lower than their levels in a normal subject. Specifically, the microbes may be at least one or more of: Akkermansia muciniphila, Clostridium butyricum, Clostridium beijerinckii, Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Eubacterium hallii, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faecis, Roseburia hominis, Roseburia intestinalis, and any combinations thereof.

Measuring the microbiome of subjects may show that microbiomes lacking various strains of microorganisms result in a health condition and/or disease state (e.g. T2DM and obesity). Restoring one or more lacking strains (e.g. via a bacterial strain such as E. hallii) results in alteration of the health condition. Some non-limiting examples include altering the gut microbiome such that the host has an increased capacity for energy harvest, increased insulin sensitivity, and/or decreased appetite.

Measuring the microbiome of subjects may be conducted by analysing body fluid samples or conducting microbial analyses on faeces of the subject. Candidate strains can be found in scientific literature and studies. These candidate strains may be used as parameters to be measured from subject's microbiome analyses. By comparing ideal levels of each microbe in a healthy individual to the levels of that microbe in the subject, a conclusion can be made regarding the levels of that microbe in the gut of the subject. Accordingly, the compositions as disclosed herein, may be administered to replenish and reinstate the population of the deficient gut microbe as identified by the microbial analyses.

For instance, as alluded above, it is known according to multiple scientific literature that lower than normal levels of E. hallii in the gut of a subject may be linked to disorders like Type-2-diabetes or weight gain. On determining the level of E. hallii in the gut of the subject and the amount by which it is lower than desirable levels, the amount and dosage of the compositions as presented in this disclosure may be administered to the subject to bring the E. hallii levels back to its desired level as in normal healthy subjects.

V. Dosing

The appropriate quantity of a therapeutic composition to be administered, the number of treatments, and unit dose may vary according to a subject and/or the disease state of the subject.

Pharmaceutical compositions described herein may be in unit dosage forms suitable for single administration of precise dosages. In unit dosage form, the formulation may be divided into unit doses containing appropriate quantities of one or more microbial compositions. The unit dosage may be in the form of a package containing discrete quantities of the formulation. Non-limiting examples are liquids in vials or ampoules. Aqueous suspension compositions may be packaged in single-dose non-recloseable containers. The composition may be in a multi-dose format. Multiple-dose recloseable containers may be used, for example, in combination with a preservative. Formulations for parenteral injection may be presented in unit dosage form, for example, in ampoules, or in multi-dose containers with a preservative.

The dosage may be in the form of a solid, semi-solid, or liquid composition. Non-limiting examples of dosage forms include feed, food, pellet, lozenge, liquid, elixir, aerosol, inhalant, spray, powder, tablet, pill, capsule, gel, geltab, nanosuspension, nanoparticle, microgel, suppository troches, aqueous or oily suspensions, ointment, patch, lotion, dentifrice, emulsion, creams, drops, dispersible powders or granules, emulsion in hard or soft gel capsules, syrups, phytoceuticals, nutraceuticals, dietary supplement, and any combination thereof.

A microbe may be present in any suitable concentration in a pharmaceutical composition. The concentration of a microbe may be for example, from about 10\hat{ }1 to about 10\hat{ }18 CFU or AFU per gram of the media. The concentration of a microbe may be, for example, at least 10\hat{ }1, at least 10\hat{ }2, at least 10\hat{ }3, at least 10\hat{ }4, at least 10\hat{ }5, at least 10\hat{ }6, at least 10\hat{ }7, at least 10\hat{ }8, at least 10\hat{ }9, at least 10\hat{ }10, at least 10\hat{ }11, at least 10\hat{ }12, at least 10\hat{ }13, at least 10\hat{ }14, at least 10\hat{ }15, at least 10\hat{ }16, at least 10\hat{ }17, or at least 10\hat{ }18 CFU or AFU per gram. The concentration of a microbe may be, for example, at most 10\hat{ }1, at most 10\hat{ }2, at most 10\hat{ }3, at most 10\hat{ }4, at most 10\hat{ }5, at most 10\hat{ }6, at most 10\hat{ }7, at most 10\hat{ }8, at most 10\hat{ }9, at most 10\hat{ }10, at most 10\hat{ }11, at most 10\hat{ }12, at most 10\hat{ }13, at most 10\hat{ }14, at most 10\hat{ }15, at most 10\hat{ }16, at most 10\hat{ }17, or at most 10\hat{ }18 CFU or AFU per gram. In some embodiments, the concentration of a microbe is from about 10\hat{ }8 CFU to about 10\hat{ }9 CFU or AFU per gram. In some embodiments, the concentration of a microbe is about 10\hat{ }8 CFU or AFU per gram. In some embodiments, the concentration of a microbe is about 10\hat{ }9 CFU or AFU per gram.

Pharmaceutical compositions, as presented here, may be formulated with any suitable therapeutically-effective concentration of prebiotic. For example, the therapeutically effective concentration of a prebiotic may be at least about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic may be at most about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. For example, the therapeutically-effective concentration of a prebiotic may be about 1 mg/ml, about 2 mg/ml, about 3 mg/ml, about 4 mg/ml, about 5 mg/ml, about 10 mg/ml, about 15 mg/ml, about 20 mg/ml, about 25 mg/ml, about 30 mg/ml, about 35 mg/ml, about 40 mg/ml, about 45 mg/ml, about 50 mg/ml, about 55 mg/ml, about 60 mg/ml, about 65 mg/ml, about 70 mg/ml, about 75 mg/ml, about 80 mg/ml, about 85 mg/ml, about 90 mg/ml, about 95 mg/ml, about 100 mg/ml, about 110 mg/ml, about 125 mg/ml, about 130 mg/ml, about 140 mg/ml, or about 150 mg/ml. In some embodiments, the concentration of a prebiotic in a pharmaceutical composition is about 70 mg/ml. In some embodiments, the prebiotic is inulin.

Pharmaceutical compositions, as in the present disclosure, may be administered for example, 1, 2, 3, 4, 5, or more times daily. In an embodiment, the pharmaceutical compositions may be administered, for example, daily, every other day, three times a week, twice a week, once a week, or at other appropriate intervals for treatment of the condition.

While the foregoing invention has been described in some detail for purposes of clarity and understanding, it will be clear to one skilled in the art from a reading of this disclosure that various changes in form and detail may be made without departing from the true scope of the invention. For example, all the compositions and methods described above may be used in various combinations. All publications, patents, patent applications, and/or other documents cited in this application are incorporated by reference in their entirety for all purposes to the same extent as if each individual publication, patent, patent application, and/or other document were individually and separately indicated to be incorporated by reference for all purposes.

### G. EXAMPLES

### Example 1: Making EHAL-ALE

Objective: The purpose is to prepare EHAL-ALE from Eubacterium hallii

Methods: Eubacterium hallii DSM 3353 is streaked on a solid PYG-agar medium bought from Anaerobe Systems and left for colonisation for 24-48 h at 37° C. After at least 24 hours, a colony of E. hallii is randomly picked and suspended in liquid PYG-Veg media as shown in Table 1 with added vitamins as shown in Table 2 and allowed to grow at 37° C. The bacteria along with the media is then centrifuged and pelleted to get the bacteria removed from the supernatant. The pellet is then cryopreserved with 5% (w/w) lactate and trehalose cryoprotectant, flash frozen, and lyophilized in LabConco FreeZone 2.5Plus lyophilizer for 8-24 h at −84° C. and a vacuum pressure of 0.008 to 0.1 mbar. The lyophilized bacteria in a proportion of ˜0.01 mg/mL is then revived by putting the lyophilized cells in liquid PYG medium (as mentioned above) and allowing the culture to grow for 12-20 h.

The suspended bacteria is then repeatedly taken through the same process of cultivation, centrifuging, pelleting, cryopreserving, lyophilizing, and re-suspending in liquid media for 22 cycles. At the end of 22 cycles, the bacteria is plated and a colony picked. That colony is EHAL-ALE.

### Example 2: Increased Viability of EHAL-ALE

Objective: The purpose of the study is to assess and compare the viability of Eubacterium hallii and Ehal-ALE.

Methods: The EHAL-ALE as obtained by the process described in Example 1, and Eubacterium hallii DSM 3353 are both allowed to grow on separate plates on PYG-agar (AS-822) liquid media for 24 hours. At the end of 24 hours, 3 colonies of each EHAL-ALE and DSM 3353 were streaked on liquid PYG media and are grown independently at 37 C. Each of these organisms are grown to a similar OD (OD600), and mixed with cryoprotectant and lyophilized as in Example 1. Viability is then measured using Flow Cytometry protocol in a BD Accuri C6 Flow Cytometer, as is well known to a person of ordinary skill in the art.

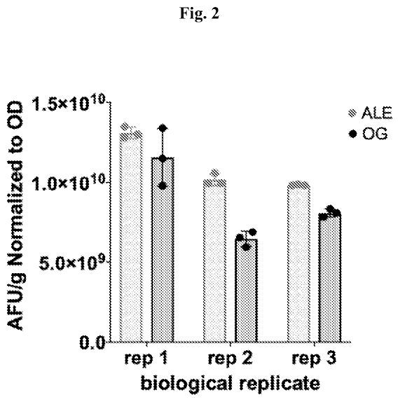

Results: Viability comparison between two Eubacterium hallii strains showing that the E. hallii DSM 3353 strain has lower viability compared with the EHAL-ALE strain. As is shown in FIG. 2, each of the three paired EHAL-ALE and DSM 3353 comparisons (named rep 1, rep 2, and rep 3) are measured in triplicate. For each one of the readings taken, EHAL-ALE has at least 25% greater viability, on average, compared to E. hallii DSM 3353. The y-axis represents Active Fluorescence Units per gram (AFU/g) resulting from a flow cytometry measurement that reports on cell wall integrity. The values have been normalized by an optical density (OD) measure in order to match cell concentrations at harvest.

### Example 3: Faster Revival EHAL-ALE Vs. Eubacterium hallii

Objective: The purpose of the study is to assess and compare the revivability characteristic of E.hallii 3353 and Ehal-ALE.

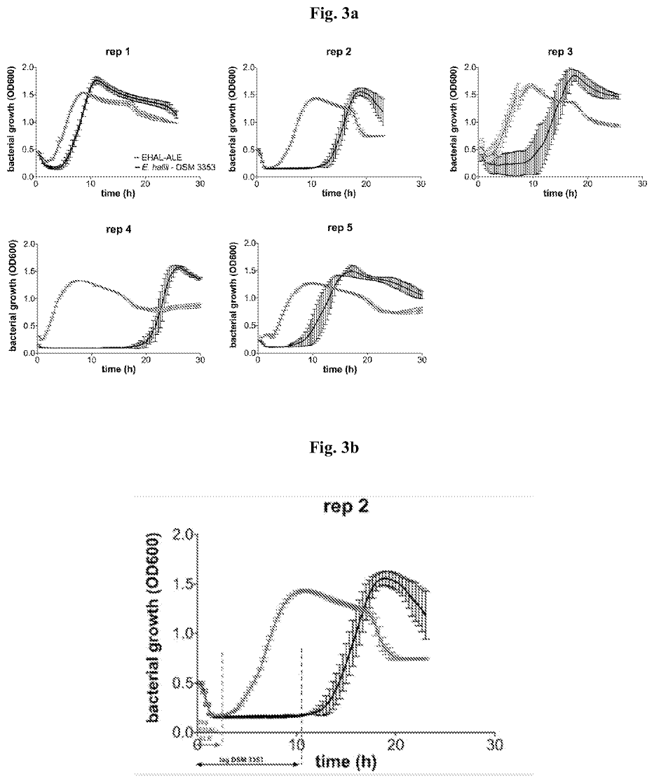

Methods: Five independent replicate samples (1-2 mg each) of lyophilized and cryoprotectant treated EHAL-ALE as in Example 1 and E.hallii DSM 3353, are each resuspended in 1 ml of PYG at time t0. As shown in FIG. 3a, the 5 samples (rep 1, rep 2, rep 3, rep 4, rep 5) of each EHAL-ALE and E. hallii DSM 3353 are each incubated at 37 C for 24-30 h. Within this time period, at regular intervals of 20 min, the bacterial growth in the PYG medium is measured, via OD600 (optical density at 600 nm). The plots of FIG. 3a show the growth in the Y-axis vs. time (measured in hours) on the Y-axis of both the OG strain and the ALE strain. The lag time is defined as the time, in hours, from inoculation (time t0) until the start of exponential growth phase (when OD600 begins to increase exponentially, with time), which can also be deciphered from each of the plots of FIG. 3a. For e.g., for rep 2, the lag phase and time associated can be marked as shown in FIG. 3b. Revivability is defined as the time it takes from inoculation to get to the start of exponential growth phase. It can also be termed as a measure of lag time. It can be noted from the FIGS. 3a and 3b that the lag phase is shorter for the ALE strain than the OG strain, across all 5 independent replicates.

### Example 4: Treatment of Type-2-Diabetes Using a Composition Comprising EHAL-ALE

Objective: The purpose of the study is to assess the effect of microbial compositions as described herein for treating Type-2-diabetes Mellitus or Type-1-diabetes mellitus.

Methods: Twenty subjects with Type-2-diabetes Mellitus, enter a double-blind, placebo controlled and randomized study.

Experimental group: Ten subjects are given oral compositions containing the active composition comprising: EHAL-ALE, Akkermansia muciniphila, Clostridium butyricum, and Clostridium beijerinckii. The composition is taken once a day for 3 weeks before meals. Parameters observed include glucose tolerance before and after administration of the composition daily for 3 weeks.

Control group: Ten subjects are given a placebo pill. The placebo is taken once a day for 3 weeks. Parameters observed include glucose tolerance before and after administration of the composition daily for 3 weeks.

Predicted Results: Following treatment, subjects in the experimental group have a

restored gut microbiome, increased glucose tolerance compared with the control group, and lower hbA1c levels than control group subjects.

### Example 5: Treatment of Obesity with a Composition Comprising EHAL-ALE

Method: A subject with obesity is prescribed a microbial-based oral composition comprising the EHAL-ALE and Akkermansia muciniphila. Each strain is present in a range of about 10\hat{ }7 to about 10\hat{ }12 CFU in the composition. The composition additionally comprises a prebiotic at a concentration of about 70 mg/mL. The expected delivery form of the oral composition is an enteric-coated (e.g., pH sensitive polymer Eudragit FS30D) pill that can protect against stomach acidity and deliver to the ileum/upper colon region of the subject. The enteric coating is designed to dissolve at a pH greater than about 6.5-7.

The subject is administered the composition before food intake (e.g., 1 hour before meals) twice daily for fourteen consecutive days.

Result: The subject loses weight and has a lower BMI than what he had before taking the prescribed oral composition.

SEQUENCE LISTING

<210> Seq. ID NO. : 1
<211> 126
<212> DNA
<213> Eubacterium hallii
<400> 1
atcattaata tgacaaaaag caacgaaatt tataaaaaaa gaggggagaa cttctatttt 60
tacccccccc cttaaaaaat tacatttttc agtgttcttt cttatatttt aactatttat 120
caatta 126
<210> 2
<211> 125
<212> DNA
<213> Eubacterium hallii
<400> 2
atcattaata tgacaaaaag caacgaaatt tataaaaaaa gaggggagaa cttctatttt 60
tacccccccc ttaaaaaatt acatttttca gtgttctttc ttatatttta actatttatc 120
aatta 125
<210> 3
<211> 144
<212> DNA
<213> Eubacterium hallii
<400> 3
atgaaaaata aaaaaatgaa tgatattttt aaaaatcttc ttgacaatta tttattaata 60
agttatcata taaacaataa caattactat tattacaaaa tggtaacaaa gggtaattac 120
ttatacatgg atactattgt atag 144
<210> 4
<211> 138
<212> DNA
<213> Eubacterium hallii
<400> 4
atgaaaaata aaaaaatgaa tgatattttt aaaaatcttc ttgacaatta tttattaata 60
agttatcata taaacaatta ctattattac aaaatggtaa caaagggtaa ttacttatac 120
atggatacta ttgtatag 138
<210> 5
<211> 1383
<212> DNA
<213> Eubacterium hallii
<400> 5
atgttagaaa aattctttaa gttatccgaa aatcacacgg atgcgaagac ggaaattctg 60
gctggtatta cgacatttat gacaatggca tatattttgg cggttaatcc aagtattatg 120
gctgctaccg gtatggattc cggtgctgta tttactgcga ctgctcttgc tgcttttatc 180
ggaactttgc tgatggcgat tttcgcgaac tatccatttg ctctggcacc gggtatggga 240
ctgaatgcat actttgccta tactgttgta atcggtatgg gatatacctg gcagtatgca 300
ctaactgctg tatttgcaga aggtatcatt tttattcttc tttctcttac aaatgtgcgt 360
gaggcgattt ttaatgcaat tccgatgaat ctgaaatctg ccgttagtgt gggaattggt 420
ctttttattg catttgttgg tttacagaat gcacatatcg ttgtgggcgg ttctacatta 480
ctccagttat tttctgtaga tgcatacaat aaagcgaatg gtgtggaagc ttcttttaac 540
aatgtcggaa ttacggtaat tcttgcattg gcaggtatta tcattaccgg aattttagtt 600
gttaaaaata ttaagggaaa tatcctctgg ggtattttaa tcacatgggg acttggaatc 660
atttgccagt ttgcaggact ttatgtaccg aatgcagatt taggattcta cagtctttta 720
cctgacttta gcaaagggct ttctattcca agtcttactc ctatctttgg aaagcttcag 780
tttaagggaa ttttctctgt agactttatc gtaattttat ttgcattttt atttgttgac 840
ctttttgata caatcggaac acttgttggt gtatctgcca aagcagatat gttagatgaa 900
gaaggaaagc ttcctcatat taaaggagca cttcttgctg atgctgtagc gactactttt 960
ggtgcaatcc tcggaacttc taccactact acttttgtgg aaagtgcttc tggagtatcc 1020
gaaggaggaa gaacaggtct tactgctgtt acaacagcga tcctttttgg attatctctg 1080
ttcttatcac caatcttcct tgcgattcca tcttttgcta ccgcacctgc tttagtaatt 1140
gttggattat atatgcttag taatgttaca aacattaact ttactgatat gtccgaggct 1200
atccctgctt acgtatgtat cattgcaatg ccattcttct atagcatctc tgagggtatc 1260
tctatgggaa ttatttctta tgtagtgatt aaccttatta ctggagaggc aaaagacaag 1320
aagatcagtg cattgatgta tgtattggca atactgttca tcttaaaata catttttctg 1380
taa 1383
<210> 6
<211> 2096
<212> DNA
<213> Eubacterium hallii
<400> 6
atgttagaaa aattctttaa gttatccgaa aatcacacgg atgcgaagac ggaaattctg 60
gctggtatta cgacatttat gacaatggca tatattttgg cggttaatcc aagtattatg 120
gctgctaccg gtatggattc cggtgctgta tttactgcga ctgctcttgc tgcttttatc 180
ggaactttgc tgatggcgat tttcgcgaac tatccatttg ctctggcacc gggtatggga 240
ctgaatgcat actttgccta tactgttgta atcggtatgg gatatacctg gcagtatgca 300
ctaactgctg tatttgcaga aggtatcatt tttattcttc tttctcttac aaatgtgcgt 360
gaggcgattt ttaatgcaat tccgatgaat ctgaaatctg ccgttagtgt gggaattggt 420
ctttttattg catttgttgg tttacagaat gcacatatcg ttgtgggcgg ttctacatta 480
ctccagttat tttctgtaga tgcatacaat aaagcgaatg gtgtggaagc ttcttttaac 540
aatgtcggaa ttacggtaat tcttgcattg gcaggtatta tcattaccgg aattttagtt 600
gttaaaaata ttaagggaaa tatcctctgg ggtattttaa tcacatgggg acttggaatc 660
atttgccagt ttgcaggact ttatgtaccg aatgcagatt taggattcta caaatgttag 720
aaaaattctt taagttatcc gaaaatcaca cggatgcgaa gacggaaatt ctggctggta 780
ttacgacatt tatgacaatg gcatatattt tggcggttaa tccaagtatt atggctgcta 840
ccggtatgga ttccggtgct gtatttactg cgactgctct tgctgctttt atcggaactt 900
tgctgatggc gattttcgcg aactatccat ttgctctggc accgggtatg ggactgaatg 960
catactttgc ctatactgtt gtaatcggta tgggatatac ctggcagtat gcactaactg 1020
ctgtatttgc agaaggtatc atttttattc ttctttctct tacaaatgtg cgtgaggcga 1080
tttttaatgc aattccgatg aatctgaaat ctgccgttag tgtgggaatt ggtcttttta 1140
ttgcatttgt tggtttacag aatgcacata tcgttgtggg cggttctaca ttactccagt 1200
tattttctgt agatgcatac aataaagcga atggtgtgga agcttctttt aacaatgtcg 1260
gaattacggt aattcttgca ttggcaggta ttatcattac cggaatttta gttgttaaaa 1320
atattaaggg aaatatcctc tggggtattt taatcacatg gggacttgga atcatttgcc 1380
agtttgcagg actttatgta ccgaatgcag atttaggatt ctacagtctt ttacctgact 1440
ttagcaaagg gctttctatt ccaagtctta ctcctatctt tggaaagctt cagtttaagg 1500
gaattttctc tgtagacttt atcgtaattt tatttgcatt tttatttgtt gacctttttg 1560
atacaatcgg aacacttgtt ggtgtatctg ccaaagcaga tatgttagat gaagaaggaa 1620
agcttcctca tattaaagga gcacttcttg ctgatgctgt agcgactact tttggtgcaa 1680
tcctcggaac ttctaccact actacttttg tggaaagtgc ttctggagta tccgaaggag 1740
gaagaacagg tcttactgct gttacaacag cgatcctttt tggattatct ctgttcttat 1800
caccaatctt ccttgcgatt ccatcttttg ctaccgcacc tgctttagta attgttggat 1860
tatatatgct tagtaatgtt acaaacatta actttactga tatgtccgag gctatccctg 1920
cttacgtatg tatcattgca atgccattct tctatagcat ctctgagggt atctctatgg 1980
gaattatttc ttatgtagtg attaacctta ttactggaga ggcaaaagac aagaagatca 2040
gtgcattgat gtatgtattg gcaatactgt tcatcttaaa atacattttt ctgtaa 2096
<210> 7

<211> 669
<212> DNA
<213> Eubacterium hallii
<400> 7

atgaaagaca ctaactttaa tgtaaatgaa tatctgccac ttcgtgacgt tgtatttaac 60
acacttcgcc aggcaattat tactggtgaa ttcgcaccgg gagaacgtct catggaaatt 120
tctcttgcca accgcctcgg tgtaagccgt actcctgtaa gagaagctat tcgtaagtta 180
gaactggaag gcctggttat catgattcca cgaaaaggtg ctcaagtagc cagaatcaca 240
gaaaaaaatc ttcgtgatgt catcgaaatc cgtactgttc ttgaagaatt cgctgctgtt 300
cttgcctgcg aacgaattga tcagtctggg cttcatgatt tacgtcaggc tcatgaggac 360
ttcatccgtt ccgtagagaa tggggatatt ctcgatatcg tagataaaga tgagactttc 420
catgatacca ttttccgcgc aacaaacaat gaccgcctta tttctattat caataatctg 480
cgcgaacagt tctatcgtta ccgcatggaa tatgtgaaag atattcgcca gcgctctaat 540
cttgtggagg aacatcggga attgcttgat gccatttcca gcagagattc tataaaggca 600
aaggaactga tgaaaacaca tctgcttaat cagcagcagg aagtaattaa taacattcag 660
gaagcatag 669

<210> 8
<211> 668
<212> DNA
<213> Eubacterium hallii
<400> 8

atgaaagaca ctaactttaa tgtaaatgat atctgccact tcgtgacgtt gtatttaaca 60
cacttcgcca ggcaattatt actggtgaat tcgcaccggg agaacgtctc atggaaattt 120
ctcttgccaa ccgcctcggt gtaagccgta ctcctgtaag agaagctatt cgtaagttag 180
aactggaagg cctggttatc atgattccac gaaaaggtgc tcaagtagcc agaatcacag 240
aaaaaaatct tcgtgatgtc atcgaaatcc gtactgttct tgaagaattc gctgctgttc 300
ttgcctgcga acgaattgat cagtctgggc ttcatgattt acgtcaggct catgaggact 360
tcatccgttc cgtagagaat ggggatattc tcgatatcgt agataaagat gagactttcc 420
atgataccat tttccgcgca acaaacaatg accgccttat ttctattatc aataatctgc 480
gcgaacagtt ctatcgttac cgcatggaat atgtgaaaga tattcgccag cgctctaatc 540
ttgtggagga acatcgggaa ttgcttgatg ccatttccag cagagattct ataaaggcaa 600
aggaactgat gaaaacacat ctgcttaatc agcagcagga agtaattaat aacattcagg 660
aagcatag 668

<210> 9
<211> 345
<212> DNA
<213> Eubacterium hallii
<400> 9

atgagagtcg tagactccga ctttcttccc caaatcttcc ccagattctt tattttggtt 60
cgtctgtccc cactacccca caaatcaatc ctagtgcatt ctgcacaaaa aaatagtcac 120
aactttattg tatttgccga aatgatgcaa atggagcaaa aaatagttga ctttttctca 180
ataaaggtat atcttagaat caagatttgg atacaaaatc aaatatcaaa aatcataaat 240
cagaaacgca aaagcaaaac attaagtaaa gaaattggga ttttcattta ctgggtatgg 300
gaggctggaa agggtaagag gatttatgat aaaagcaaaa cgtaa 345
<210> 10
<211> 345
<212> DNA
<213> Eubacterium hallii

<400> 10

atgagagtcg tagactccga ctttcttccc caaatcttcc ccagattctt tattttggtt 60
cgtctgtccc cactacccca caaatcaatc ctagtgcatt ctgcacaaaa aaatagtcac 120
aactttattg tatttgccga aatgatgcaa atggagcaaa aaatagttga ctttttctca 180
ataaaggtat atcttagaat caagatttga atacaaaatc aaatatcaaa aatcataaat 240
cagaaacgca aaagcaaaac attaagtaaa gaaattggga ttttcattta ctgggtatgg 300
gaggctggaa agggtaagag gatttatgat aaaagcaaaa cgtaa 345

<210> 11
<211> 1989
<212> DNA
<213> Eubacterium hallii
<400> 11

atggaacgtt tgatttattt tgcaccggtt cttggaatct gcgcattgct ttttgcgttt 60
tatcttacaa aaaaagtagg aaaacaggat gcaggaacag atagaatgaa ggagattgca 120
gcatttattc atgagggagc aagagctttc cttactgcag aatataagat tcttgtcgtt 180
tttgttgctg tattatttgt actgattggt attggtatcg gtaactgggt aactgcagta 240
tgtttcttag tcggagcatt attttccact gcagccggtt atatcggaat gaacgttgcg 300
actaaggcga atgtaagaac agccgcagcg gcaaaagata gcggaatgaa taaagcttta 360
tctatcgcat tttccggtgg tgccgttatg ggtatgtgtg tagtaggttt tggacttttt 420
ggagcaggtg ttgtatatat cctgacaaaa aatccggatg ttctttctgg tttctcttta 480
ggagcttctt ctatcgcatt gtttgcccgt gtaggtggcg gtatctatac aaaggctgcc 540
gacgttggag cggatctcgt aggtaaagta gaagcaggta tcccggaaga tgatcctcgt 600
aacccagctg ttatcgctga taacgtaggt gacaacgtag gtgatgttgc cggaatgggt 660
gctgatttat ttgagtctta tgtagggtct ttagtatctg ccatcactct tggtgttgtt 720
tacgcaaaag agagcggagc tattttccca ttagttattg cagcacttgg tgtattagca 780
tctgttattg gatgtttctt cgtaaagggt gatgagaatt ccagcccgca caaggcattg 840
aaatacggaa gttattctgc agcaatcgtg gtaatgattg gttctttaat tttaagcaaa 900
atgttcttca acggattcaa agaagctatc gcgattatct ttggtcttgt tgttggtctt 960
ttaatcggtg taatcacaga gatttatacg tctggagatt accgttttgt taaaaaaatc 1020
gcacagcagt ctgagactgg tcctgcaaca acagttatca gtggtattgc cgtaggtatg 1080
cagtccacag ccgttccgat tattttaatc gctatcggaa tcattggggc atattctttc 1140
agcggtcttt acggaattgc tttggcagca gtaggtatgc tctccacaac aggtattacg 1200
gtagccgtag atgcttacgg tccaatcgca gataacgctg gtggtattgc tgagatgtct 1260
ggccttccat cagaagttcg taacatcaca gacaaattag acgccgttgg taacacaaca 1320
gccgctatgg gtaaaggttt cgctatcggt tctgcagccc ttaccgcatt agcattattc 1380
gtatcttacg cacaggcagt tggattattc gaagaaggaa tcaacttatt agattataaa 1440
gtaattgtag gtatgtttgt aggtggaatg cttccattcc tcttctccgc atttacaatg 1500
gattctgtat ccaaagcagc atataagatg attgaagaag taagaagaca gtttaaaaca 1560
attcctggaa tcttagaagg aaaaggaaag ccagattata aatcctgcgt agcaatctct 1620
acacaggcag cattaaaaga aatgcttctc ccaggtgtta tggcagtact tgcaccagta 1680
tttatcggcg ttgtacttgg acctgatgcg cttggcggac ttcttggcgg tgcattagta 1740
acaggtgtta tgctcgcgat tttcatgtca aactccggtg gagcatggga taatgcgaag 1800
aaatacattg aagatggaca tcacggcgga aagggaagcg aagcacatcg tgcggcagta 1860
gttggtgata cagtaggtga tccattcaag gatacttcag gaccttctat taatatcctt 1920
attaagctga tgacaattgt atcattggta tttgcaccat tgttcctcaa aatcggagga 1980
ttattctaa 1989
<210> 12

<211> 1989
<212> DNA
<213> Eubacterium hallii
<400> 12

atggaacgtt tgatttattt tgcaccggtt cttggaatct gcgcattgct ttttgcgttt 60
tatcttacaa aaaaagtagg aaaacaggat gcaggaacag atagaatgaa ggagattgca 120
gcatttattc atgagggagc aagagctttc cttactgcag aatataagat tcttgtcgtt 180
tttgttgctg tattatttgt actgattggt attggtatcg gtaactgggt aactgcagta 240
tgtttcttag tcggagcatt attttccact gcagccggtt atatcggaat gaacgttgcg 300
actaaggcga atgtaagaac agccgcagcg gcaaaagata gcggaatgaa taaagcttta 360
tctatcgcat tttccggtgg tgccgttatg ggtatgtgtg tagtaggttt tggacttttt 420
ggagcaggtg ttgtatatat cctgacaaaa aatccggatg ttctttctgg tttctcttta 480
ggagcttctt ctatcgcatt gtttgcccgt gtaggtggcg gtatctatac aaaggctgcc 540
gacgttggag cggatctcgt aggtaaagta gaagcaggta tcccggaaga tgatcctcgt 600
aacccagctg ttatcgctga taacgtaggt gacaacgtag gtgatgttgc cggaatgggt 660
gctgatttat ttgagtctta tgtagggtct ttagtatctg ccatcactct tggtgttgtt 720
tacgcaaaag agagtggagc tattttccca ttagttattg cagcacttgg tgtattagca 780
tctgttattg gatgtttctt cgtaaagggt gatgagaatt ccagcccgca caaggcattg 840
aaatacggaa gttattctgc agcaatcgtg gtaatgattg gttctttaat tttaagcaaa 900
atgttcttca acggattcaa agaagctatc gcgattatct ttggtcttgt tgttggtctt 960
ttaatcggtg taatcacaga gatttatacg tctggagatt accgttttgt taaaaaaatc 1020
gcacagcagt ctgagactgg tcctgcaaca acagttatca gtggtattgc cgtaggtatg 1080
cagtccacag ccgttccgat tattttaatc gctatcggaa tcattggggc atattctttc 1140
agcggtcttt acggaattgc tttggcagca gtaggtatgc tctccacaac aggtattacg 1200
gtagccgtag atgcttacgg tccaatcgca gataacgctg gtggtattgc tgagatgtct 1260
ggccttccat cagaagttcg taacatcaca gacaaattag acgccgttgg taacacaaca 1320
gccgctatgg gtaaaggttt cgctatcggt tctgcagccc ttaccgcatt agcattattc 1380
gtatcttacg cacaggcagt tggattattc gaagaaggaa tcaacttatt agattataaa 1440
gtaattgtag gtatgtttgt aggtggaatg cttccattcc tcttctccgc atttacaatg 1500
gattctgtat ccaaagcagc atataagatg attgaagaag taagaagaca gtttaaaaca 1560
attcctggaa tcttagaagg aaaaggaaag ccagattata aatcctgcgt agcaatctct 1620
acacaggcag cattaaaaga aatgcttctc ccaggtgtta tggcagtact tgcaccagta 1680
tttatcggcg ttgtacttgg acctgatgcg cttggcggac ttcttggcgg tgcattagta 1740
acaggtgtta tgctcgcgat tttcatgtca aactccggtg gagcatggga taatgcgaag 1800
aaatacattg aagatggaca tcacggcgga aagggaagcg aagcacatcg tgcggcagta 1860
gttggtgata cagtaggtga tccattcaag gatacttcag gaccttctat taatatcctt 1920
attaagctga tgacaattgt atcattggta tttgcaccat tgttcctcaa aatcggagga 1980
ttattctaa 1989
<210> 13
<211> 1479
<212> DNA
<213> Eubacterium hallii
<400> 13
atgaacagtt cgattattcg ttttgtatta ggatatgtat tgaagatgga agcagtgtta 60
atgcttcttc cttgtattgt tgctgtgctc taccgggagc agacaggttt tgcctatctt 120
ttagtggcag cggtatctat ggcatttggt acattgatga cgataaagaa acccaaaagt 180
catgtgtttt atttaaaaga gggctgtgta gcaacggctt taagctggat atttttaagt 240
ttttttggtg cactgccatt ctggatttct ggagaaattc catctttgat cgatgcttta 300
tttgagacag tttctggttt tacgacaacc ggttcaagta ttttagcaga tgtggaagca 360
ctttctcatt gtgctttatt ctggagaagc tttactcact ggattggcgg tatgggagta 420
ctggtattcc ttcttgcggt tattccgtta agtggaggtt ctcatattaa cttaatgcgt 480
gcagaaagtc cgggaccatc tgtaggaaag cttgtgccaa agattaaata cactgcacag 540
attctctata ttatttattt tggaatgaca attgtagaga ttgtgcttct tcttatcagc 600
aggatgcctg catttgatgc gattactctg tcgtttggaa cggcaggtac cggtggcttt 660
ggtataaagg gagacagtct tgccagttat acggcactgc agcagtggat tgtcactata 720
tttatgattt tatttggagt aaacttcaac gcttattatt tgattttatt tagaaagttc 780
aaaaaagcac ttcaaatgga ggaagtacgc gcatactttg ctattatttt cgtggcaaca 840
gcaattatta ccggtagttt agtcggagga aatatgcagt tttttgatgc acttcgtcat 900
gccgcattcc aagtgggttc tatcattaca acaacaggat atgcaaccgt caattttgat 960
gcttggtcgc agacttgccg ggcaattctt gtccttttga tgtttgtggg agcctgtgca 1020
ggaagtactg gtggaggaat aaaagtatct cgttttattg ttatggtaaa gacaatgatt 1080
aaggaactca attcctacat ccatccaaag agtgtaaaga agattaaaat ggatgacaaa 1140
ccaatagagc atgaagtagt acgctccatt aatgtatatt ttattacctt tatgattatt 1200
tttgtcgctt cagtcatagc catttcattt gaaggccatg atcttgtaac gaactttact 1260
gctatagcag cgacaatcaa caatattggt ccggggctta gtatggttgg tccggactgt 1320
aactttggat tcttcagtga tttttccaag cttgttatta tttttgatat gcttgcagga 1380
agactggaat tattcccgtt acttatttta ttccatccag cgatttggaa ggaattgttt 1440
acgcagaaaa tcacgatgag gaggaagaag aaattctga 1479
<210> 14
<211> 1479
<212> DNA
<213> Eubacterium hallii
<400> 14
atgaacagtt cgattattcg ttttgtatta ggatatgtat tgaagatgga agcagtgtta 60
atgcttcttc cttgtattgt tgctgtgctc taccgggagc agacaggttt tgcctatctt 120
ttagtggcag cggtatctat ggcatttggt acattgatga cgataaagaa acccaaaagt 180
catgtgtttt atttaaaaga gggctgtgta gcaacggctt taagctggat atttttaagt 240
ttttttggtg cactgccatt ctggatttct ggagaaattc catctttgat cgatgcttta 300
tttgagacag tttctggttt tacgacaacc ggttcaagta ttttagcaga tgtggaagca 360
ctttctcatt gtgctttatt ctggagaagc tttactcact ggattggcgg tatgggagta 420
ctggtattcc ttcttgcggt tattccgtta agtggaggtt ctcatattaa cttaatgcgt 480
gcagaaagtc cgggaccatc tgtaggaaag cttgtgccaa agattaaata cactgcacag 540
attctctata ttatttattt tggaatgaca attgtagaga ttgtgcttct tcttatcagc 600
aggatgcctg catttgatgc gattactctg tcgtttggaa cggcaggtac cggtggcttt 660
ggtataaagg gagacagtct tgccagttat acggcactgc agcagtggat tgtcactata 720
tttatgattt tatttggagt aaacttcaac gcttattatt tgattttatt tagaaagttc 780
aaaaaagcac ttcaaatgga ggaagtacgc gcatactttg ctattatttt cgtggcaaca 840
gcaattatta ccggtagttt agtcggagga aatatgcagt tttttgatgc acttcgtcat 900
gccgcattcc aagtgggttc tatcattaca acaacaggat atgcaaccgt caattttgat 960
gcttggtcgc agacttgccg ggcaattctt gtccttttga tgtttgtggg agcctgtgca 1020
ggaagtactg gtggaggaat aaaagtatct cgttttattg ttatggtaaa gacaatgatt 1080
aaggaactca attcctacat ccatccaaag agtgtaaaga agattaaaat ggatgacaaa 1140
ccaatagagc atgaagtagt acgctccatt aatgtatatt ttattacctt tatgattatt 1200
tttgtcgctt cagtcatagc catttcattt gaaggccatg atcttgtaac gaactttact 1260
gctatagcag cgacaatcaa caatattggt ccggggctta gtatggttgg tccggactgt 1320
aactttggat tcttcagtga tttttccaag cttgttatta tttttgatat gcttgcagaa 1380
agactggaat tattcccgtt acttatttta ttccatccag cgatttggaa ggaattgttt 1440
acgcagaaaa tcacgatgag gaggaagaag aaattctga 1479

What is claimed is:

1. A method of isolating a bacteria having increased viability when revived from lyophilization, the method comprising:
culturing the bacteria in anaerobic conditions in/on a vegetable-based growth medium;
lyophilizing the bacteria from the culture in the presence of a cryoprotectant;
reviving the lyophilized bacteria;
determining that the revived bacteria has increased viability;
isolating the revived bacteria with the increased viability; and
wherein the increased viability is as compared to a wild-type bacteria being revived from lyophilization.

2. The method according to claim 1, wherein culturing the bacteria comprises culturing the bacteria to obtain least 1×10\hat{ }5 AFUs/g of the culture.

3. The method according to claim 1, wherein culturing the bacteria comprises measuring the growth of the bacteria at regular time intervals.

4. The method according to claim 3, wherein the regular time intervals are every 10 minutes, every 20 minutes, every 30 minutes, every 1 hour, every 4 hours, or every 6 hours.

5. The method according to claim 1, wherein the vegetable-based medium is essentially free of any animal or dairy components or derivatives thereof.

6. The method according to claim 1, wherein the vegetable-based medium comprises peptone, yeast extract, and glucose.

7. The method according to claim 1, wherein the bacterium is as deposited at ATCC® accession number 126295.

8. The method according to claim 1, wherein the bacterium comprises at least one of SEQ ID No., 2, 4, 6, 8, 10, 12 or 14.

9. The method according to claim 1, wherein the bacterium comprises at least one of:
a. a deletion of C at a position corresponding to any one of positions 63 to 71 of SEQ ID No. 1;
b. a deletion of AACAAT at a position corresponding to positions 79 to 84 of SEQ ID No. 3;
c. a replacement of G with an A at a position corresponding to position 713 of SEQ ID No. 5;
d. a deletion of A at a position corresponding to any one of positions 29 to 30 of SEQ ID No. 7;
e. a replacement of G with an A at a position corresponding to any one of positions 209 or 210 of SEQ ID No. 9;
f. a replacement of C with a G at a position corresponding to position 735 of SEQ ID No. 11; or
g. a replacement of G with an A at a position corresponding to any one of positions 1378 or 1379 of SEQ ID No. 13.

10. A bacteria as deposited at ATCC accession number 126295.

11. A bacteria comprising at least one of SEQ ID No., 2, 4, 6, 8, 10, 12 or 14.

12. A bacteria comprising at least one of:
a. a deletion of C at a position corresponding to any one of positions 63 to 71 of SEQ ID No. 1;
b. a deletion of AACAAT at a position corresponding to positions 79 to 84 of SEQ ID No. 3;
c. a replacement of G with an A at a position corresponding to position 713 of SEQ ID No. 5;
d. a deletion of A at a position corresponding to any one of positions 29 to 30 of SEQ ID No. 7;
e. a replacement of G with an A at a position corresponding to any one of positions 209 or 210 of SEQ ID No. 9;
f. a replacement of C with a G at a position corresponding to position 735 of SEQ ID No. 11; or
g. a replacement of G with an A at a position corresponding to any one of positions 1378 or 1379 of SEQ ID No. 13.

13. The bacteria of any one of claims 1-12, wherein the bacteria has at least 95% 16s rDNA sequence homology to the full length 16s rRNA of any one of Eubacterium hallii DSM 3353, Akkermansia muciniphila ATCC BAA83, Clostridium butyricum ATCC 1939, Clostridium beijerinckii ATCC 5174 or combinations thereof.

14. The bacteria of any one of claims 1-12, wherein the bacteria is Eubacterium hallii.

15. The bacteria of any one of claims 1-12, wherein the bacteria belongs to the genus Clostridia, Eubacteria, Bifidobacteria, Anaerostipes, Coprococcus, Bacteroides, Blautia, Ruminococcus, Faecalibacterium, Oscillospira, Streptococcus, or Roseburiai.

16. The bacteria of any one of claims 1-12, wherein the bacteria is selected from the group consisting of Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus eutactus, Eubacterium cylindroides, Eubacterium dolichum, Eubacterium ventriosum, Roseburia faeccis, Roseburia hominis, Roseburia intestinalis, and all combinations thereof.

17. A composition comprising any one or more of bacteria as in claims 1 and 10-16.

18. The composition of claim 19, wherein the composition comprises at least 10\hat{ }5 AFUs/g of each of the one or more additional bacteria.

19. The composition of claim 17 or claim 18, further comprising a prebiotic.

20. The composition of claim 19, wherein the prebiotic is selected from the group consisting of inulin, green banana, reishi, tapioca, oats, pectin, potato or extracts thereof.

21. The composition of claim 18, wherein at least one of the one or more additional bacteria is an anaerobic bacteria.

22. The composition of claims 17-18 and 21, wherein the bacteria is lyophilized.

23. The composition of claims 17-18 and 21, wherein the composition comprises at least 10\hat{ }5 AFUs/g of the bacteria.

24. The composition of claims 17-18 and 21, wherein the bacteria is not viable or is dead.

25. The composition of claims 17-18 and 21, wherein the composition is in the form of a pill, a capsule, a lozenge, a food bar, or a gummy ball.

26. The composition of claims 17-18 and 21, wherein the bacteria is pasteurized.

27. A method of treating a subject, the method comprising administering to the subject the bacteria of any one of claims 1-12.

28. The method according to claim 27, further comprising administering to the subject at least one another anaerobic bacteria.

29. The method according to claim 27 or 28, wherein the subject suffers from a gut disorder.

30. The method according to claim 29, wherein the gut disorder is irritable bowel syndrome, inflammatory bowel disease, a stomach ulcer, pouchitis, Helicobacter pylori infection, or diarrhea.

31. The method according to claim 27 or 28, wherein the subject suffers from, or has a risk of, an insulin resistance-based disorder or an insulin sensitivity-based disorder.

32. The method according to claim 31, wherein the subject suffers from, or has a risk of, Type-2-diabetes.

33. The method of claim 27-32, wherein the subject is a mammal.

34. The method of claim 33, wherein the mammal is a human.

35. The method of claim 27 or 28, wherein the administration is an oral administration.

36. A composition comprising a bacteria having a revival rate that is at least 25% faster than E. hallii deposited at DSM 3353, wherein the bacteria has at least 97% sequence identity to the full length of a 16S rRNA sequence of E. hallii as deposited at DSM 3353, and wherein the revival rate is measured by an assay comprising the steps of:
a. providing a lyophilized bacteria and a lyophilized E. hallii deposited at DSM 3353;
b. suspending the lyophilized bacteria and the lyophilized E. hallii in a vegetable medium;
c. incubating the suspension for at least 24 hours in 37° C.;
d. measuring bacterial growth in regular time intervals of at least 5 minutes at an OD600; and
e. determining revival rate of the bacteria and the E. hallii deposited as DSM 3353 based on the measured bacterial growth in step (d) herein.

37. The bacteria of claim 36, wherein the bacteria is deposited at ATCC® with an accession number 126295.

38. The composition of claim 36, wherein the bacteria comprises at least one of SEQ ID No., 2, 4, 6, 8, 10, 12 or 14.

39. The composition of claim 36, wherein the bacteria comprises at least one of:
a. a deletion of C at a position corresponding to any one of positions 63 to 71 of SEQ ID No. 1;
b. a deletion of AACAAT at a position corresponding to positions 79 to 84 of SEQ ID No. 3;
c. a replacement of G with an A at a position corresponding to position 713 of SEQ ID No. 5;
d. a deletion of A at a position corresponding to any one of positions 29 to 30 of SEQ ID No. 7;
e. a replacement of G with an A at a position corresponding to any one of positions 209 or 210 of SEQ ID No. 9;
f. a replacement of C with a G at a position corresponding to position 735 of SEQ ID No. 11; or
g. a replacement of G with an A at a position corresponding to any one of positions 1378 or 1379 of SEQ ID No. 13.

40. The vegetable media of claim 36, wherein the media is free of any animal product or dairy product or derivatives thereof.

41. The composition of claim 36, wherein the media is a PYG media further comprising salt, buffer, and vitamins.

42. The composition of claim 36, further comprising one or more additional bacterial strains.

43. The composition of claim 42, wherein the one or more additional bacteria strains are selected from the group consisting of Akkermansia muciniphila, Clostridium butyricum, Clostridium beijerinckii, and all combinations thereof.

44. The composition of claim 42, wherein the one or more additional bacteria strains are selected from the group consisting of Akkermansia muciniphila ATCC BAA83, Clostridium butyricum ATCC 1939, Clostridium beijerinckii ATCC 5174 or combinations thereof.

45. The composition of claim 42, wherein the one or more additional bacteria strains are selected from the group consisting of Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and all combinations thereof.

46. The composition of claim 42, wherein each of the one or more additional bacteria strains has a 16S rRNA sequence comprising at least about 95% sequence identity to the full length of a 16S rRNA sequence of a microbe selected from the group consisting of: Anaerostipes caccae, Bifidobacterium adolescentis, Bifidobacterium bifidum, Bifidobacterium infantis, Bifidobacterium longum, Butyrivibrio fibrisolvens, Clostridium acetobutylicum, Clostridium aminophilum, Clostridium beijerinckii, Clostridium butyricum, Clostridium colinum, Clostridium indolis, Clostridium orbiscindens, Enterococcus faecium, Eubacterium hallii, Eubacterium rectale, Faecalibacterium prausnitzii, Fibrobacter succinogenes, Oscillospira guilliermondii, Roseburia cecicola, Roseburia inulinivorans, Ruminococcus flavefaciens, Ruminococcus gnavus, Ruminococcus obeum, Streptococcus cremoris, Streptococcus faecium, Streptococcus infantis, Streptococcus mutans, Streptococcus thermophilus, Anaerofustis stercorihominis, Anaerostipes hadrus, Anaerotruncus colihominis, Clostridium sporogenes, Clostridium tetani, Coprococcus, Coprococcus eutactus, Eubacterium cylindroides, Eubacterium dolichum, Eubacterium ventriosum, Roseburia faeccis, Roseburia hominis, Roseburia intestinalis, and any combination thereof.

47. The composition of claim 42, wherein the one or more additional bacteria strains belong to the genus of Clostridia, Eubacteria, Bifidobacteria, Anaerostipes, Coprococcus, Bacteroides, Blautia, Ruminococcus, Faecalibacterium, Oscillospira, Streptococcus, or Roseburiail.

48. The composition of claim 42, wherein the composition comprises at least 10\hat{ }5 AFUs/g of each of the one or more additional bacteria.

49. The composition of claim 36, further comprising a prebiotic.

50. The composition of claim 49, wherein the prebiotic is selected from the group consisting of inulin, green banana, reishi, tapioca, oats, pectin, potato or extracts thereof.

51. The composition of claim 42, wherein at least one of the one or more additional bacteria is an anaerobic bacteria.

52. The composition of claim 42, wherein the composition comprises at least 10\hat{ }5 AFUs/g of the bacteria.

53. The composition of claim 36, wherein the composition is in the form of a pill, a capsule, a lozenge, a food bar, or a gummy ball.

54. The composition of claim 36, wherein the composition further comprises an enteric coating.

55. The composition of claim 54, wherein the enteric coating is Eudragit FS30D.


SEQUENCE LISTING (21 sequences)

SEQ ID NO: 1 - DNA, 126 bp, Eubacterium hallii
atcattaatatgacaaaaagcaacgaaatttataaaaaaagaggggagaacttctatttttacccccccccttaaaaaattacatttttcagtgttctttcttatattttaactatttatcaatta

SEQ ID NO: 2 - DNA, 125 bp, Eubacterium hallii
atcattaatatgacaaaaagcaacgaaatttataaaaaaagaggggagaacttctatttttaccccccccttaaaaaattacatttttcagtgttctttcttatattttaactatttatcaatta

SEQ ID NO: 3 - DNA, 144 bp, Eubacterium hallii
atgaaaaataaaaaaatgaatgatatttttaaaaatcttcttgacaattatttattaataagttatcatataaacaataacaattactattattacaaaatggtaacaaagggtaattacttatacatggatactattgtatag

SEQ ID NO: 4 - DNA, 138 bp, Eubacterium hallii
atgaaaaataaaaaaatgaatgatatttttaaaaatcttcttgacaattatttattaataagttatcatataaacaattactattattacaaaatggtaacaaagggtaattacttatacatggatactattgtatag

SEQ ID NO: 5 - DNA, 1383 bp, Eubacterium hallii
atgttagaaaaattctttaagttatccgaaaatcacacggatgcgaagacggaaattctggctggtattacgacatttatgacaatggcatatattttggcggttaatccaagtattatggctgctaccggtatggattccggtgctgtatttactgcgactgctcttgctgcttttatcggaactttgctgatggcgattttcgcgaactatccatttgctctggcaccgggtatgggactgaatgcatactttgcctatactgttgtaatcggtatgggatatacctggcagtatgcactaactgctgtatttgcagaaggtatcatttttattcttctttctcttacaaatgtgcgtgaggcgatttttaatgcaattccgatgaatctgaaatctgccgttagtgtgggaattggtctttttattgcatttgttggtttacagaatgcacatatcgttgtgggcggttctacattactccagttattttctgtagatgcatacaataaagcgaatggtgtggaagcttcttttaacaatgtcggaattacggtaattcttgcattggcaggtattatcattaccggaattttagttgttaaaaatattaagggaaatatcctctggggtattttaatcacatggggacttggaatcatttgccagtttgcaggactttatgtaccgaatgcagatttaggattctacagtcttttacctgactttagcaaagggctttctattccaagtcttactcctatctttggaaagcttcagtttaagggaattttctctgtagactttatcgtaattttatttgcatttttatttgttgacctttttgatacaatcggaacacttgttggtgtatctgccaaagcagatatgttagatgaagaaggaaagcttcctcatattaaaggagcacttcttgctgatgctgtagcgactacttttggtgcaatcctcggaacttctaccactactacttttgtggaaagtgcttctggagtatccgaaggaggaagaacaggtcttactgctgttacaacagcgatcctttttggattatctctgttcttatcaccaatcttccttgcgattccatcttttgctaccgcacctgctttagtaattgttggattatatatgcttagtaatgttacaaacattaactttactgatatgtccgaggctatccctgcttacgtatgtatcattgcaatgccattcttctatagcatctctgagggtatctctatgggaattatttcttatgtagtgattaaccttattactggagaggcaaaagacaagaagatcagtgcattgatgtatgtattggcaatactgttcatcttaaaatacatttttctgtaa

SEQ ID NO: 6 - DNA, 2096 bp, Eubacterium hallii
atgttagaaaaattctttaagttatccgaaaatcacacggatgcgaagacggaaattctggctggtattacgacatttatgacaatggcatatattttggcggttaatccaagtattatggctgctaccggtatggattccggtgctgtatttactgcgactgctcttgctgcttttatcggaactttgctgatggcgattttcgcgaactatccatttgctctggcaccgggtatgggactgaatgcatactttgcctatactgttgtaatcggtatgggatatacctggcagtatgcactaactgctgtatttgcagaaggtatcatttttattcttctttctcttacaaatgtgcgtgaggcgatttttaatgcaattccgatgaatctgaaatctgccgttagtgtgggaattggtctttttattgcatttgttggtttacagaatgcacatatcgttgtgggcggttctacattactccagttattttctgtagatgcatacaataaagcgaatggtgtggaagcttcttttaacaatgtcggaattacggtaattcttgcattggcaggtattatcattaccggaattttagttgttaaaaatattaagggaaatatcctctggggtattttaatcacatggggacttggaatcatttgccagtttgcaggactttatgtaccgaatgcagatttaggattctacaaatgttagaaaaattctttaagttatccgaaaatcacacggatgcgaagacggaaattctggctggtattacgacatttatgacaatggcatatattttggcggttaatccaagtattatggctgctaccggtatggattccggtgctgtatttactgcgactgctcttgctgcttttatcggaactttgctgatggcgattttcgcgaactatccatttgctctggcaccgggtatgggactgaatgcatactttgcctatactgttgtaatcggtatgggatatacctggcagtatgcactaactgctgtatttgcagaaggtatcatttttattcttctttctcttacaaatgtgcgtgaggcgatttttaatgcaattccgatgaatctgaaatctgccgttagtgtgggaattggtctttttattgcatttgttggtttacagaatgcacatatcgttgtgggcggttctacattactccagttattttctgtagatgcatacaataaagcgaatggtgtggaagcttcttttaacaatgtcggaattacggtaattcttgcattggcaggtattatcattaccggaattttagttgttaaaaatattaagggaaatatcctctggggtattttaatcacatggggacttggaatcatttgccagtttgcaggactttatgtaccgaatgcagatttaggattctacagtcttttacctgactttagcaaagggctttctattccaagtcttactcctatctttggaaagcttcagtttaagggaattttctctgtagactttatcgtaattttatttgcatttttatttgttgacctttttgatacaatcggaacacttgttggtgtatctgccaaagcagatatgttagatgaagaaggaaagcttcctcatattaaaggagcacttcttgctgatgctgtagcgactacttttggtgcaatcctcggaacttctaccactactacttttgtggaaagtgcttctggagtatccgaaggaggaagaacaggtcttactgctgttacaacagcgatcctttttggattatctctgttcttatcaccaatcttccttgcgattccatcttttgctaccgcacctgctttagtaattgttggattatatatgcttagtaatgttacaaacattaactttactgatatgtccgaggctatccctgcttacgtatgtatcattgcaatgccattcttctatagcatctctgagggtatctctatgggaattatttcttatgtagtgattaaccttattactggagaggcaaaagacaagaagatcagtgcattgatgtatgtattggcaatactgttcatcttaaaatacatttttctgtaa

SEQ ID NO: 7 - DNA, 669 bp, Eubacterium hallii
atgaaagacactaactttaatgtaaatgaatatctgccacttcgtgacgttgtatttaacacacttcgccaggcaattattactggtgaattcgcaccgggagaacgtctcatggaaatttctcttgccaaccgcctcggtgtaagccgtactcctgtaagagaagctattcgtaagttagaactggaaggcctggttatcatgattccacgaaaaggtgctcaagtagccagaatcacagaaaaaaatcttcgtgatgtcatcgaaatccgtactgttcttgaagaattcgctgctgttcttgcctgcgaacgaattgatcagtctgggcttcatgatttacgtcaggctcatgaggacttcatccgttccgtagagaatggggatattctcgatatcgtagataaagatgagactttccatgataccattttccgcgcaacaaacaatgaccgccttatttctattatcaataatctgcgcgaacagttctatcgttaccgcatggaatatgtgaaagatattcgccagcgctctaatcttgtggaggaacatcgggaattgcttgatgccatttccagcagagattctataaaggcaaaggaactgatgaaaacacatctgcttaatcagcagcaggaagtaattaataacattcaggaagcatag

SEQ ID NO: 8 - DNA, 668 bp, Eubacterium hallii
atgaaagacactaactttaatgtaaatgatatctgccacttcgtgacgttgtatttaacacacttcgccaggcaattattactggtgaattcgcaccgggagaacgtctcatggaaatttctcttgccaaccgcctcggtgtaagccgtactcctgtaagagaagctattcgtaagttagaactggaaggcctggttatcatgattccacgaaaaggtgctcaagtagccagaatcacagaaaaaaatcttcgtgatgtcatcgaaatccgtactgttcttgaagaattcgctgctgttcttgcctgcgaacgaattgatcagtctgggcttcatgatttacgtcaggctcatgaggacttcatccgttccgtagagaatggggatattctcgatatcgtagataaagatgagactttccatgataccattttccgcgcaacaaacaatgaccgccttatttctattatcaataatctgcgcgaacagttctatcgttaccgcatggaatatgtgaaagatattcgccagcgctctaatcttgtggaggaacatcgggaattgcttgatgccatttccagcagagattctataaaggcaaaggaactgatgaaaacacatctgcttaatcagcagcaggaagtaattaataacattcaggaagcatag

SEQ ID NO: 9 - DNA, 345 bp, Eubacterium hallii
atgagagtcgtagactccgactttcttccccaaatcttccccagattctttattttggttcgtctgtccccactaccccacaaatcaatcctagtgcattctgcacaaaaaaatagtcacaactttattgtatttgccgaaatgatgcaaatggagcaaaaaatagttgactttttctcaataaaggtatatcttagaatcaagatttggatacaaaatcaaatatcaaaaatcataaatcagaaacgcaaaagcaaaacattaagtaaagaaattgggattttcatttactgggtatgggaggctggaaagggtaagaggatttatgataaaagcaaaacgtaa

SEQ ID NO: 10 - DNA, 345 bp, Eubacterium hallii
atgagagtcgtagactccgactttcttccccaaatcttccccagattctttattttggttcgtctgtccccactaccccacaaatcaatcctagtgcattctgcacaaaaaaatagtcacaactttattgtatttgccgaaatgatgcaaatggagcaaaaaatagttgactttttctcaataaaggtatatcttagaatcaagatttgaatacaaaatcaaatatcaaaaatcataaatcagaaacgcaaaagcaaaacattaagtaaagaaattgggattttcatttactgggtatgggaggctggaaagggtaagaggatttatgataaaagcaaaacgtaa

SEQ ID NO: 11 - DNA, 1989 bp, Eubacterium hallii
atggaacgtttgatttattttgcaccggttcttggaatctgcgcattgctttttgcgttttatcttacaaaaaaagtaggaaaacaggatgcaggaacagatagaatgaaggagattgcagcatttattcatgagggagcaagagctttccttactgcagaatataagattcttgtcgtttttgttgctgtattatttgtactgattggtattggtatcggtaactgggtaactgcagtatgtttcttagtcggagcattattttccactgcagccggttatatcggaatgaacgttgcgactaaggcgaatgtaagaacagccgcagcggcaaaagatagcggaatgaataaagctttatctatcgcattttccggtggtgccgttatgggtatgtgtgtagtaggttttggactttttggagcaggtgttgtatatatcctgacaaaaaatccggatgttctttctggtttctctttaggagcttcttctatcgcattgtttgcccgtgtaggtggcggtatctatacaaaggctgccgacgttggagcggatctcgtaggtaaagtagaagcaggtatcccggaagatgatcctcgtaacccagctgttatcgctgataacgtaggtgacaacgtaggtgatgttgccggaatgggtgctgatttatttgagtcttatgtagggtctttagtatctgccatcactcttggtgttgtttacgcaaaagagagcggagctattttcccattagttattgcagcacttggtgtattagcatctgttattggatgtttcttcgtaaagggtgatgagaattccagcccgcacaaggcattgaaatacggaagttattctgcagcaatcgtggtaatgattggttctttaattttaagcaaaatgttcttcaacggattcaaagaagctatcgcgattatctttggtcttgttgttggtcttttaatcggtgtaatcacagagatttatacgtctggagattaccgttttgttaaaaaaatcgcacagcagtctgagactggtcctgcaacaacagttatcagtggtattgccgtaggtatgcagtccacagccgttccgattattttaatcgctatcggaatcattggggcatattctttcagcggtctttacggaattgctttggcagcagtaggtatgctctccacaacaggtattacggtagccgtagatgcttacggtccaatcgcagataacgctggtggtattgctgagatgtctggccttccatcagaagttcgtaacatcacagacaaattagacgccgttggtaacacaacagccgctatgggtaaaggtttcgctatcggttctgcagcccttaccgcattagcattattcgtatcttacgcacaggcagttggattattcgaagaaggaatcaacttattagattataaagtaattgtaggtatgtttgtaggtggaatgcttccattcctcttctccgcatttacaatggattctgtatccaaagcagcatataagatgattgaagaagtaagaagacagtttaaaacaattcctggaatcttagaaggaaaaggaaagccagattataaatcctgcgtagcaatctctacacaggcagcattaaaagaaatgcttctcccaggtgttatggcagtacttgcaccagtatttatcggcgttgtacttggacctgatgcgcttggcggacttcttggcggtgcattagtaacaggtgttatgctcgcgattttcatgtcaaactccggtggagcatgggataatgcgaagaaatacattgaagatggacatcacggcggaaagggaagcgaagcacatcgtgcggcagtagttggtgatacagtaggtgatccattcaaggatacttcaggaccttctattaatatccttattaagctgatgacaattgtatcattggtatttgcaccattgttcctcaaaatcggaggattattctaa

SEQ ID NO: 12 - DNA, 1989 bp, Eubacterium hallii
atggaacgtttgatttattttgcaccggttcttggaatctgcgcattgctttttgcgttttatcttacaaaaaaagtaggaaaacaggatgcaggaacagatagaatgaaggagattgcagcatttattcatgagggagcaagagctttccttactgcagaatataagattcttgtcgtttttgttgctgtattatttgtactgattggtattggtatcggtaactgggtaactgcagtatgtttcttagtcggagcattattttccactgcagccggttatatcggaatgaacgttgcgactaaggcgaatgtaagaacagccgcagcggcaaaagatagcggaatgaataaagctttatctatcgcattttccggtggtgccgttatgggtatgtgtgtagtaggttttggactttttggagcaggtgttgtatatatcctgacaaaaaatccggatgttctttctggtttctctttaggagcttcttctatcgcattgtttgcccgtgtaggtggcggtatctatacaaaggctgccgacgttggagcggatctcgtaggtaaagtagaagcaggtatcccggaagatgatcctcgtaacccagctgttatcgctgataacgtaggtgacaacgtaggtgatgttgccggaatgggtgctgatttatttgagtcttatgtagggtctttagtatctgccatcactcttggtgttgtttacgcaaaagagagtggagctattttcccattagttattgcagcacttggtgtattagcatctgttattggatgtttcttcgtaaagggtgatgagaattccagcccgcacaaggcattgaaatacggaagttattctgcagcaatcgtggtaatgattggttctttaattttaagcaaaatgttcttcaacggattcaaagaagctatcgcgattatctttggtcttgttgttggtcttttaatcggtgtaatcacagagatttatacgtctggagattaccgttttgttaaaaaaatcgcacagcagtctgagactggtcctgcaacaacagttatcagtggtattgccgtaggtatgcagtccacagccgttccgattattttaatcgctatcggaatcattggggcatattctttcagcggtctttacggaattgctttggcagcagtaggtatgctctccacaacaggtattacggtagccgtagatgcttacggtccaatcgcagataacgctggtggtattgctgagatgtctggccttccatcagaagttcgtaacatcacagacaaattagacgccgttggtaacacaacagccgctatgggtaaaggtttcgctatcggttctgcagcccttaccgcattagcattattcgtatcttacgcacaggcagttggattattcgaagaaggaatcaacttattagattataaagtaattgtaggtatgtttgtaggtggaatgcttccattcctcttctccgcatttacaatggattctgtatccaaagcagcatataagatgattgaagaagtaagaagacagtttaaaacaattcctggaatcttagaaggaaaaggaaagccagattataaatcctgcgtagcaatctctacacaggcagcattaaaagaaatgcttctcccaggtgttatggcagtacttgcaccagtatttatcggcgttgtacttggacctgatgcgcttggcggacttcttggcggtgcattagtaacaggtgttatgctcgcgattttcatgtcaaactccggtggagcatgggataatgcgaagaaatacattgaagatggacatcacggcggaaagggaagcgaagcacatcgtgcggcagtagttggtgatacagtaggtgatccattcaaggatacttcaggaccttctattaatatccttattaagctgatgacaattgtatcattggtatttgcaccattgttcctcaaaatcggaggattattctaa

SEQ ID NO: 13 - DNA, 1479 bp, Eubacterium hallii
atgaacagttcgattattcgttttgtattaggatatgtattgaagatggaagcagtgttaatgcttcttccttgtattgttgctgtgctctaccgggagcagacaggttttgcctatcttttagtggcagcggtatctatggcatttggtacattgatgacgataaagaaacccaaaagtcatgtgttttatttaaaagagggctgtgtagcaacggctttaagctggatatttttaagtttttttggtgcactgccattctggatttctggagaaattccatctttgatcgatgctttatttgagacagtttctggttttacgacaaccggttcaagtattttagcagatgtggaagcactttctcattgtgctttattctggagaagctttactcactggattggcggtatgggagtactggtattccttcttgcggttattccgttaagtggaggttctcatattaacttaatgcgtgcagaaagtccgggaccatctgtaggaaagcttgtgccaaagattaaatacactgcacagattctctatattatttattttggaatgacaattgtagagattgtgcttcttcttatcagcaggatgcctgcatttgatgcgattactctgtcgtttggaacggcaggtaccggtggctttggtataaagggagacagtcttgccagttatacggcactgcagcagtggattgtcactatatttatgattttatttggagtaaacttcaacgcttattatttgattttatttagaaagttcaaaaaagcacttcaaatggaggaagtacgcgcatactttgctattattttcgtggcaacagcaattattaccggtagtttagtcggaggaaatatgcagttttttgatgcacttcgtcatgccgcattccaagtgggttctatcattacaacaacaggatatgcaaccgtcaattttgatgcttggtcgcagacttgccgggcaattcttgtccttttgatgtttgtgggagcctgtgcaggaagtactggtggaggaataaaagtatctcgttttattgttatggtaaagacaatgattaaggaactcaattcctacatccatccaaagagtgtaaagaagattaaaatggatgacaaaccaatagagcatgaagtagtacgctccattaatgtatattttattacctttatgattatttttgtcgcttcagtcatagccatttcatttgaaggccatgatcttgtaacgaactttactgctatagcagcgacaatcaacaatattggtccggggcttagtatggttggtccggactgtaactttggattcttcagtgatttttccaagcttgttattatttttgatatgcttgcaggaagactggaattattcccgttacttattttattccatccagcgatttggaaggaattgtttacgcagaaaatcacgatgaggaggaagaagaaattctga

SEQ ID NO: 14 - DNA, 1479 bp, Eubacterium hallii
atgaacagttcgattattcgttttgtattaggatatgtattgaagatggaagcagtgttaatgcttcttccttgtattgttgctgtgctctaccgggagcagacaggttttgcctatcttttagtggcagcggtatctatggcatttggtacattgatgacgataaagaaacccaaaagtcatgtgttttatttaaaagagggctgtgtagcaacggctttaagctggatatttttaagtttttttggtgcactgccattctggatttctggagaaattccatctttgatcgatgctttatttgagacagtttctggttttacgacaaccggttcaagtattttagcagatgtggaagcactttctcattgtgctttattctggagaagctttactcactggattggcggtatgggagtactggtattccttcttgcggttattccgttaagtggaggttctcatattaacttaatgcgtgcagaaagtccgggaccatctgtaggaaagcttgtgccaaagattaaatacactgcacagattctctatattatttattttggaatgacaattgtagagattgtgcttcttcttatcagcaggatgcctgcatttgatgcgattactctgtcgtttggaacggcaggtaccggtggctttggtataaagggagacagtcttgccagttatacggcactgcagcagtggattgtcactatatttatgattttatttggagtaaacttcaacgcttattatttgattttatttagaaagttcaaaaaagcacttcaaatggaggaagtacgcgcatactttgctattattttcgtggcaacagcaattattaccggtagtttagtcggaggaaatatgcagttttttgatgcacttcgtcatgccgcattccaagtgggttctatcattacaacaacaggatatgcaaccgtcaattttgatgcttggtcgcagacttgccgggcaattcttgtccttttgatgtttgtgggagcctgtgcaggaagtactggtggaggaataaaagtatctcgttttattgttatggtaaagacaatgattaaggaactcaattcctacatccatccaaagagtgtaaagaagattaaaatggatgacaaaccaatagagcatgaagtagtacgctccattaatgtatattttattacctttatgattatttttgtcgcttcagtcatagccatttcatttgaaggccatgatcttgtaacgaactttactgctatagcagcgacaatcaacaatattggtccggggcttagtatggttggtccggactgtaactttggattcttcagtgatttttccaagcttgttattatttttgatatgcttgcagaaagactggaattattcccgttacttattttattccatccagcgatttggaaggaattgtttacgcagaaaatcacgatgaggaggaagaagaaattctga

SEQ ID NO: 15 - DNA, 126 bp, Artificial Sequence; identical to SEQ ID NO: 1
  Description of Artificial Sequence Synthetic polynucleotide
  misc_feature (63)..(71): May or may not be present
  See specification as filed for detailed description of substitutions and preferred embodiments

SEQ ID NO: 16 - DNA, 138 bp, Artificial Sequence; identical to SEQ ID NO: 4
  Description of Artificial Sequence Synthetic polynucleotide

SEQ ID NO: 17 - DNA, 1383 bp, Artificial Sequence
  Description of Artificial Sequence Synthetic polynucleotide
atgttagaaaaattctttaagttatccgaaaatcacacggatgcgaagacggaaattctggctggtattacgacatttatgacaatggcatatattttggcggttaatccaagtattatggctgctaccggtatggattccggtgctgtatttactgcgactgctcttgctgcttttatcggaactttgctgatggcgattttcgcgaactatccatttgctctggcaccgggtatgggactgaatgcatactttgcctatactgttgtaatcggtatgggatatacctggcagtatgcactaactgctgtatttgcagaaggtatcatttttattcttctttctcttacaaatgtgcgtgaggcgatttttaatgcaattccgatgaatctgaaatctgccgttagtgtgggaattggtctttttattgcatttgttggtttacagaatgcacatatcgttgtgggcggttctacattactccagttattttctgtagatgcatacaataaagcgaatggtgtggaagcttcttttaacaatgtcggaattacggtaattcttgcattggcaggtattatcattaccggaattttagttgttaaaaatattaagggaaatatcctctggggtattttaatcacatggggacttggaatcatttgccagtttgcaggactttatgtaccgaatgcagatttaggattctacaatcttttacctgactttagcaaagggctttctattccaagtcttactcctatctttggaaagcttcagtttaagggaattttctctgtagactttatcgtaattttatttgcatttttatttgttgacctttttgatacaatcggaacacttgttggtgtatctgccaaagcagatatgttagatgaagaaggaaagcttcctcatattaaaggagcacttcttgctgatgctgtagcgactacttttggtgcaatcctcggaacttctaccactactacttttgtggaaagtgcttctggagtatccgaaggaggaagaacaggtcttactgctgttacaacagcgatcctttttggattatctctgttcttatcaccaatcttccttgcgattccatcttttgctaccgcacctgctttagtaattgttggattatatatgcttagtaatgttacaaacattaactttactgatatgtccgaggctatccctgcttacgtatgtatcattgcaatgccattcttctatagcatctctgagggtatctctatgggaattatttcttatgtagtgattaaccttattactggagaggcaaaagacaagaagatcagtgcattgatgtatgtattggcaatactgttcatcttaaaatacatttttctgtaa

SEQ ID NO: 18 - DNA, 669 bp, Artificial Sequence; identical to SEQ ID NO: 7
  Description of Artificial Sequence Synthetic polynucleotide
  misc_feature (29)..(30): May or may not be present
  See specification as filed for detailed description of substitutions and preferred embodiments

SEQ ID NO: 19 - DNA, 345 bp, Artificial Sequence
  Description of Artificial Sequence Synthetic polynucleotide
  See specification as filed for detailed description of substitutions and preferred embodiments
atgagagtcgtagactccgactttcttccccaaatcttccccagattctttattttggttcgtctgtccccactaccccacaaatcaatcctagtgcattctgcacaaaaaaatagtcacaactttattgtatttgccgaaatgatgcaaatggagcaaaaaatagttgactttttctcaataaaggtatatcttagaatcaagatttrratacaaaatcaaatatcaaaaatcataaatcagaaacgcaaaagcaaaacattaagtaaagaaattgggattttcatttactgggtatgggaggctggaaagggtaagaggatttatgataaaagcaaaacgtaa

SEQ ID NO: 20 - DNA, 1989 bp, Artificial Sequence
  Description of Artificial Sequence Synthetic polynucleotide
atggaacgtttgatttattttgcaccggttcttggaatctgcgcattgctttttgcgttttatcttacaaaaaaagtaggaaaacaggatgcaggaacagatagaatgaaggagattgcagcatttattcatgagggagcaagagctttccttactgcagaatataagattcttgtcgtttttgttgctgtattatttgtactgattggtattggtatcggtaactgggtaactgcagtatgtttcttagtcggagcattattttccactgcagccggttatatcggaatgaacgttgcgactaaggcgaatgtaagaacagccgcagcggcaaaagatagcggaatgaataaagctttatctatcgcattttccggtggtgccgttatgggtatgtgtgtagtaggttttggactttttggagcaggtgttgtatatatcctgacaaaaaatccggatgttctttctggtttctctttaggagcttcttctatcgcattgtttgcccgtgtaggtggcggtatctatacaaaggctgccgacgttggagcggatctcgtaggtaaagtagaagcaggtatcccggaagatgatcctcgtaacccagctgttatcgctgataacgtaggtgacaacgtaggtgatgttgccggaatgggtgctgatttatttgagtcttatgtagggtctttagtatctgccatcactcttggtgttgtttacgcaaaagagaggggagctattttcccattagttattgcagcacttggtgtattagcatctgttattggatgtttcttcgtaaagggtgatgagaattccagcccgcacaaggcattgaaatacggaagttattctgcagcaatcgtggtaatgattggttctttaattttaagcaaaatgttcttcaacggattcaaagaagctatcgcgattatctttggtcttgttgttggtcttttaatcggtgtaatcacagagatttatacgtctggagattaccgttttgttaaaaaaatcgcacagcagtctgagactggtcctgcaacaacagttatcagtggtattgccgtaggtatgcagtccacagccgttccgattattttaatcgctatcggaatcattggggcatattctttcagcggtctttacggaattgctttggcagcagtaggtatgctctccacaacaggtattacggtagccgtagatgcttacggtccaatcgcagataacgctggtggtattgctgagatgtctggccttccatcagaagttcgtaacatcacagacaaattagacgccgttggtaacacaacagccgctatgggtaaaggtttcgctatcggttctgcagcccttaccgcattagcattattcgtatcttacgcacaggcagttggattattcgaagaaggaatcaacttattagattataaagtaattgtaggtatgtttgtaggtggaatgcttccattcctcttctccgcatttacaatggattctgtatccaaagcagcatataagatgattgaagaagtaagaagacagtttaaaacaattcctggaatcttagaaggaaaaggaaagccagattataaatcctgcgtagcaatctctacacaggcagcattaaaagaaatgcttctcccaggtgttatggcagtacttgcaccagtatttatcggcgttgtacttggacctgatgcgcttggcggacttcttggcggtgcattagtaacaggtgttatgctcgcgattttcatgtcaaactccggtggagcatgggataatgcgaagaaatacattgaagatggacatcacggcggaaagggaagcgaagcacatcgtgcggcagtagttggtgatacagtaggtgatccattcaaggatacttcaggaccttctattaatatccttattaagctgatgacaattgtatcattggtatttgcaccattgttcctcaaaatcggaggattattctaa

SEQ ID NO: 21 - DNA, 1479 bp, Artificial Sequence
  Description of Artificial Sequence Synthetic polynucleotide
  See specification as filed for detailed description of substitutions and preferred embodiments
atgaacagttcgattattcgttttgtattaggatatgtattgaagatggaagcagtgttaatgcttcttccttgtattgttgctgtgctctaccgggagcagacaggttttgcctatcttttagtggcagcggtatctatggcatttggtacattgatgacgataaagaaacccaaaagtcatgtgttttatttaaaagagggctgtgtagcaacggctttaagctggatatttttaagtttttttggtgcactgccattctggatttctggagaaattccatctttgatcgatgctttatttgagacagtttctggttttacgacaaccggttcaagtattttagcagatgtggaagcactttctcattgtgctttattctggagaagctttactcactggattggcggtatgggagtactggtattccttcttgcggttattccgttaagtggaggttctcatattaacttaatgcgtgcagaaagtccgggaccatctgtaggaaagcttgtgccaaagattaaatacactgcacagattctctatattatttattttggaatgacaattgtagagattgtgcttcttcttatcagcaggatgcctgcatttgatgcgattactctgtcgtttggaacggcaggtaccggtggctttggtataaagggagacagtcttgccagttatacggcactgcagcagtggattgtcactatatttatgattttatttggagtaaacttcaacgcttattatttgattttatttagaaagttcaaaaaagcacttcaaatggaggaagtacgcgcatactttgctattattttcgtggcaacagcaattattaccggtagtttagtcggaggaaatatgcagttttttgatgcacttcgtcatgccgcattccaagtgggttctatcattacaacaacaggatatgcaaccgtcaattttgatgcttggtcgcagacttgccgggcaattcttgtccttttgatgtttgtgggagcctgtgcaggaagtactggtggaggaataaaagtatctcgttttattgttatggtaaagacaatgattaaggaactcaattcctacatccatccaaagagtgtaaagaagattaaaatggatgacaaaccaatagagcatgaagtagtacgctccattaatgtatattttattacctttatgattatttttgtcgcttcagtcatagccatttcatttgaaggccatgatcttgtaacgaactttactgctatagcagcgacaatcaacaatattggtccggggcttagtatggttggtccggactgtaactttggattcttcagtgatttttccaagcttgttattatttttgatatgcttgcarraagactggaattattcccgttacttattttattccatccagcgatttggaaggaattgtttacgcagaaaatcacgatgaggaggaagaagaaattctga